(19) United States
(12) Patent Application Publication
(10) Pub. No.: US 2004/0181094 A1
(43) Pub. Date: Sep. 16, 2004

(54) Transition metal oxo complexes as catalysts of synthetic processes involving alkyne reactants

(76) Inventor: F. Dean Toste, Berkeley, CA (US)

Correspondence Address: REED & EBERLE LLP, 800 MENLO AVENUE, SUITE 210, MENLO PARK, CA, 94025, US

(21) Appl. No.: 10/387,328

(22) Filed: Mar. 11, 2003

(51) Int. Cl. B01J 23/42; C07C 43/14

(52) U.S. Cl. 564/489; 568/687; 502/326

(57) ABSTRACT

A method is provided for synthesizing substituted alkynes from an alkyne reactant and a nucleophile using rhenium (V) oxo complex as a catalyst. The alkyne reactant is substituted at the propargylic position with a leaving group susceptible to displacement by the nucleophile in a nucleophilic substitution reaction. The method involves contacting the alkyne reactant with a nucleophilic reactant in the presence of a catalytically effective amount of the rhenium (V) oxo complex. The method does not require activation of the leaving group or ionization of the nucleophilic reactant, and may be carried out in the presence of air and moisture. The invention is useful in synthesizing propargyl ethers, propargyl amines, and the like.

### TECHNICAL FIELD

This invention relates generally to a catalytic method for the modification of alkynes, and more particularly pertains to chemical syntheses involving catalytic transformation of an alkyne reactant substituted at the propargylic position with a leaving group capable of undergoing nucleophilic displacement.

### BACKGROUND OF THE INVENTION

Alkynes are highly versatile reactants and intermediates in organic synthesis because the carbon-carbon triple bond may be readily transformed into a variety of functional groups. For example, ketones and aldehydes may be derived from alkynes by hydration, and olefins may be derived from alkynes by reduction. Methods for preparing substituted alkynes are, therefore, highly desirable in the field of synthetic organic chemistry. Processes for synthesizing alkynes substituted at the propargylic position—i.e., at the carbon atom (t to the triple bond—are particularly desirable in order to facilitate transformation of the carbon-carbon triple bond. The most commonly used method for preparing alkynes substituted at the propargylic position is the dicobalt hexacarbonyl mediated reaction of propargyl ethers and alcohols according to the Nicholas reaction, illustrated schematically in FIG. 1. See Nicholas (1987) Acc. Chew. Res. 20:207, and Martin (2000) Tetrahedron Lett. 2000:9993. The Nicholas reaction suffers from several limitations, however: (1) two equivalents of cobalt metal are required; (2) an equivalent of a strong Lewis acid is required to generate the cationic intermediate; and (3) a total of four steps are required, including a final oxidative decomplexation of the cobalt, to accomplish the substitution.

Ideally, the aforementioned reaction would be accomplished in a single step, using a very small amount of a catalyst. A preferred catalyst would be stable to air and moisture, so that precautions to avoid air and/or water contamination are unnecessary. Optimally, the catalyst would also be tolerant of a wide range of functional groups on the reactants, e.g., esters, anhydrides, olefins, and the like. It would also be desirable if the reaction could be carried out in a stereoselective (e.g., enantioselective) manner.

The present invention is directed to addressing the aforementioned need in the art, and provides a novel method for modifying alkynes that are substituted at the propargylic position with a functional group. The method is useful in a variety of reactions wherein it is desirable to form a new bond between a carbon atom on the reactant and a heteroatom of a second reactant. One exemplary use of the method is in the formation of carbon-oxygen bonds, e.g., in the synthesis of an ether.

Previously, simple alcohols have not been viable nucleophiles or electrophiles for the formation of carbon-oxygen bonds. Ether formation has typically required deprotonation of the alcohol nucleophile and a reactive electrophile, such as a halide or pseudohalide. See, e.g., Muci et al. (2002) Top. Curr. Chem. 219:131, Hartwig et al. (1998) Acc. Chem. Res. 31:852, and Prim et al. (2002) Tetrahedron 58:2041, pertaining to the formation of sp²-C—O bonds of aryl ethers from aryl halides and alcohols; and Mitsunobu, in Comprehensive Organic Synthesis, Vol 6, Trost et al., Eds. (Pergamon Press: New York, 1991), at pp 22-28. For example, formation of sp³-C—O bonds by transition metal catalyzed allylic etherfication requires the generation of copper (Evans (2002) J. Am. Chem. Soc. 124:7882; Evans et al. (2002) J. Am. Chem. Soc. 122:5012) or zinc (Kim et al. (2002) Org. Lett. 4:4369) alkoxides as nucleophiles and allylic esters or carbonates as electrophiles. A rutheniuim-catalyzed propargylic etherifcation has been reported by Nishibayashi et al. (2000) J. Am. Chem. Soc. 122:1019. That reaction, however, is limited to terminal propargyl alcohols; see Inada et al. (2002) J. Am. Chem. Soc. 124:15172.

The present invention provides a significant advance in the chemical synthesis of ethers and other reaction products resulting from a nucleophilic substitution reaction. With respect to ethers, for example, the invention provides a single-step transition metal catalyzed propargylic etherifcation reaction in which a new sp³-C—O bond is generated, using a propargylic alcohol as the substrate and a second alcohol as the nucleophile, without need for harsh reagents or activating groups.

### SUMMARY OF THE INVENTION

The present invention accordingly provides a novel method for catalyzing a nucleophilic substitution reaction between an alkyne reactant and a nucleophile, so as to provide a modified alkyne containing a newly formed carbon-heteroatom bond. The method involves contacting (a) an alkyne reactant substituted at the propargylic position with a leaving group capable of displacement by a nucleophile with (b) a nucleophilic reactant in the presence of (c) a catalytically effective amount of a rhenium (V) oxo complex having at least one electron donor ligand and at least two anionic ligands, under reaction conditions effective to provide for nucleophilic displacement of the leaving group. The alkyne reactant is represented by the structure of formula (I)

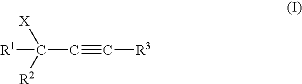

in which:

X is the leaving group;

R¹ is selected from hydrogen, C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl;

R² is selected from hydrogen, C₁-C₂₄ hydrocarbyl, substituted C₁-C₂₄ hydrocarbyl, heteroatom-containing C₁-C₂₄ hydrocarbyl, substituted heteroatom-containing C₁-C₂₄ hydrocarbyl, and functional groups; and

R³ is selected from hydrogen, silyl, C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl.

The nucleophilic reactant is a compound comprising a nucleophilic group selected from hydroxyl, hydrocarbyloxy, primary amino, secondary amino, silyl, alkenyl, aryl, and heteroaryl, any of which, with the exception of hydroxyl, may be further substituted and/or heteroatom-containing.

Preferred transition metal complexes have the structure of formula (II)

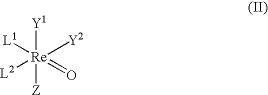

wherein:

L¹ and L² are monodentate neutral electron donor ligands, or may be taken together to form a single bidentate neutral electron donor ligand;

Y¹ and Y² are anionic ligands; and

Z is a monodentate neutral electron donor ligand or an anionic ligand.

For example, L¹, L², and Z may be independently selected from the group consisting of phosphine, sulfonated phosphine, phosphite, phosphinite, phosphonite, arsine, stibine, ether, amine, amide, imine, sulfoxide, carboxyl, nitrosyl, pyridine, substituted pyridine, imidazole, substituted imidazole, pyrazine, and thioether, or L¹ and L² may together form a bidentate ligand in which at least one coordinating heteroatom is other than N. Exemplary anionic ligands that may serve as Y¹, Y² and Z include, without limitation, hydride, halide, C₁-C₂₄ alkyl, C₅-C₂₄ aryl, C₁-C₂₄ alkoxy, C₅-C₂₄ aryloxy, C₃-C₂₄ alkyldiketonate, C₅-C₂₄ aryldiketonate, C₂-C₂₄ alkoxycarbonyl, C₅-C₂₄ aryloxycarbonyl, C₂-C₂₄ acyl, C₁-C₂₄ alkylsulfonato, C₅-C₂₄ arylsulfonato, C₁-C₂₄ alkylsulfanyl, C₅-C₂₄ arylsulfanyl, C₁-C₂₄ alkylsulfinyl, or C₅-C₂₄ arylsulfinyl, any of which, with the exception of hydride and halide, are optionally further substituted with one or more groups selected from halide, C₁-C₆ alkyl, C₁-C₆ alkoxy, and phenyl.

It will be appreciated that the metal complex of formula (II) and other complexes illustrated in a similar manner herein may have several configurations, and each possible configuration is encompassed by the generic structure. For example, formula (III) includes the metal complexes

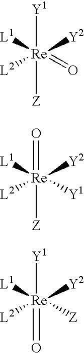

In a preferred implementation of the invention, the aforementioned method is employed for the synthesis of a propargyl ether from a propargyl alcohol and a second alcohol as nucleophile, wherein the catalyst is a complex having the structure of formula (IX)

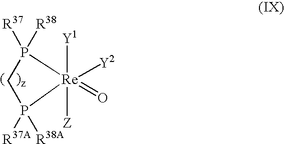

in which R³⁷, R³⁸, R^{37A}, and R^{38A} are aryl, z is 1, 2, or 3, and Y¹, Y² and Z are anionic ligands. When R³⁷, R³⁸, R^{37A}, and R^{38A} are phenyl, and Y¹, Y², and Z are chloro, it will be appreciated that the complex is (dppm)ReOCl₃, (dppe)ReOCl₃, or (dppp)ReOCl₃, depending on whether z is 1, 2, or 3, respectively, wherein “dppm” represents bis(diphenylphosphino)methane, “dppe” represents 1,2-bis(diphenylphosphino)ethane, and “dppp” represents 1,3-bis(diphenylphosphino)propane.

The metal complex of formula (III) and other complexes herein may also be used with a co-catalyst, although a co-catalyst is not required. Suitable co-catalysts are those composed of a cationic component capable of abstracting an anionic ligand from the metal complex and an anion that does not coordinate to the rhenium center. When co-catalysts are used, then, it will be appreciated that the metal complex is in the form of a cation in association with the anion of the co-catalyst.

In another embodiment, the invention provides a method for transforming a propargylic alcohol to give an enone by contacting a propargylic alcohol of formula (II) (wherein X is OH) with a catalytically effective amount of a rhenium (V) oxo complex having at least one electron donor ligand and at least two anionic ligands, under reaction conditions effective to effect rearrangement of the alcohol to provide the enone. Without being bound by theory, it appears that this rearrangement reaction proceeds through a mechanism similar to that of the nucleophilic substitution reaction, in which an intermediate is formed between the oxygen atom of the propargyl alcohol and the rhenium atom of the catalytic complex (thus displacing an anionic ligand). Rearrangement of the reactant while coupled to the catalyst will then result in the enone.

The present process is generally carried out in a polar aprotic solvent, at a temperature in the range of about 20° C. to about 80° C., using an excess of the nucleophilic reactant, i.e., the molar ratio of the nucleophilic reactant to the alkyne reactant is greater than 1:1. The amount of catalyst used can be quite small, on the order of 1 mole % or less, relative to the alkyne reactant. The synthesis may be carried out in the presence of air and moisture, so that no special precautions are necessary in this regard. The product is obtained in a single step, and the catalyst may be easily recovered by the addition of a nonpolar solvent to the crude product mixture to cause precipitation of the catalyst.

### BRIEF DESCRIPTION OF THE DRAWINGS

FIG. 1 provides a schematic illustration of a method for preparing substituted alkynes according to the prior art.

FIG. 2 schematically illustrates a synthetic method of the invention for carrying out the same transformation shown in FIG. 1.

FIG. 3 illustrates the use of the present methodology to synthesize tolterodine, starting with a propargyl arylation reaction catalyzed by a rhenium (V) oxo complex as described herein.

FIG. 4 illustrates the use of the present methodology to synthesize deoxypicropodophyllin, starting with a propargyl arylation reaction catalyzed by a rhenium (V) oxo complex as described herein.

FIG. 5 illustrates the use of the present methodology to synthesize calopin, starting with a propargylation reaction using an allylsilane as a nucleophile and catalyzed by a rhenium (V) oxo complex as described herein.

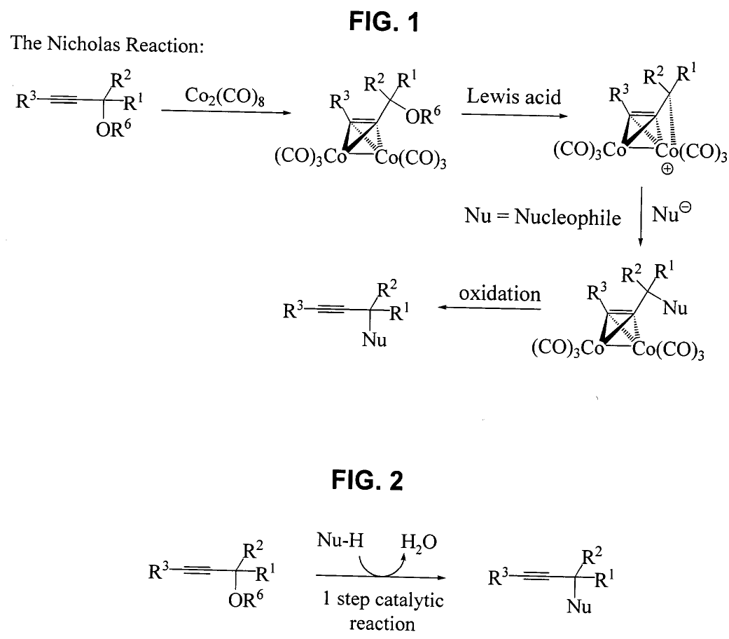

### DETAILED DESCRIPTION OF THE INVENTION

I. Definitions and Nomenclature:

It is to be understood that unless otherwise indicated this invention is not limited to specific reactants, reaction conditions, ligands, metal complexes, or the like, as such may vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a compound” encompasses a combination or mixture of different compounds as well as a single compound, reference to “a functional group” includes a single functional group as well as two or more functional groups that may or may not be the same, and the like.

In this specification and in the claims that follow, reference will be made to a number of terms, which shall be defined to have the following meanings:

As used herein, the phrase “having the formula” or “having the structure” is not intended to be limiting and is used in the same way that the term “comprising” is commonly used.

The term “alkyl” as used herein refers to a linear, branched or cyclic saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “lower alkyl” intends an alkyl group of 1 to 6 carbon atoms, preferably 1 to 4 carbon atoms, and the specific term “cycloalkyl” intends a cyclic alkyl group, typically having 4 to 8, preferably 5 to 7, carbon atoms. The term “substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl, respectively.

The term “alkenyl” as used herein refers to a linear, branched or cyclic hydrocarbon group of 2 to 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Preferred alkenyl groups herein contain 2 to 12 carbon atoms. The term “lower alkenyl” intends an alkenyl group of 2 to 6 carbon atoms, preferably 2 to 4 carbon atoms, and the specific term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Preferred alkynyl groups herein contain 2 to 12 carbon atoms. The term “lower alkynyl” intends an alkynyl group of 2 to 6 carbon atoms, preferably 2 to 4 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, preferably 1 to 4 carbon atoms. Analogously, “alkenyloxy” and “lower alkenyloxy” respectively refer to an alkenyl and lower alkenyl group bound through a single, terminal ether linkage, and “alkynyloxy” and “lower alkynyloxy” respectively refer to an alkynyl and lower alkynyl group bound through a single, terminal ether linkage.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 24 carbon atoms, and particularly preferred aryloxy groups contain 5 to 14 carbon atoms. Examples of aryloxy groups include, without limitation, phenoxy, o-halo-phenoxy, m-halo-phenoxy, p-halophenoxy, o-methoxy-phenoxy, m-methoxy-phenoxy, p-methoxy-phenoxy, 2,4-dimethoxyphenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Preferred alkaryl and aralkyl groups contain 6 to 24 carbon atoms, and particularly preferred alkaryl and aralkyl groups contain 6 to 16 carbon atoms. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like. Examples of aralkyl groups include, without limitation, benzyl, 2-phenyl-ethyl, 3-phenyl-propyl, 4-phenylbutyl, 5-phenyl-pentyl, 4-phenylcyclohexyl, 4-benzylcyclohexyl, 4-phenylcyclohexylmethyl, 4-benzylcyclohexylmethyl, and the like. The terms “alkaryloxy” and “aralkyloxy” refer to substituents of the formula —OR wherein R is alkaryl or aralkyl, respectively, as just defined.

The term “acyl” refers to substituents having the formula —(CO)-alkyl, —(CO)-aryl, or —(CO)-aralkyl, and the term “acyloxy” refers to substituents having the formula —O(CO)-alkyl, —O(CO)-aryl, or —O(CO)-aralkyl, wherein “alkyl,” “aryl, and “aralkyl” are as defined above.

The term “cyclic” refers to alicyclic or aromatic substituents that may or may not be substituted and/or heteroatom containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic or polycyclic.

The terms “halo” and “halogen” are used in the conventional sense to refer to a chloro, bromo, fluoro or iodo substituent.

“Hydrocarbyl” refers to univalent hydrocarbyl radicals containing 1 to about 30 carbon atoms, preferably 1 to about 24 carbon atoms, most preferably 1 to about 12 carbon atoms, including linear, branched, cyclic, saturated and unsaturated species, such as alkyl groups, alkenyl groups, aryl groups, and the like. The term “lower hydrocarbyl” intends a hydrocarbyl group of 1 to 6 carbon atoms, preferably 1 to 4 carbon atoms, and the term “hydrocarbylene” intends a divalent hydrocarbyl moiety containing 1 to about 30 carbon atoms, preferably 1 to about 24 carbon atoms, most preferably 1 to about 12 carbon atoms, including linear, branched, cyclic, saturated and unsaturated species. The term “lower hydrocarbylene” intends a hydrocarbylene group of 1 to 6 carbon atoms. “Substituted hydrocarbyl” refers to hydrocarbyl substituted with one or more substituent groups, and the terms “heteroatom-containing hydrocarbyl” and “heterohydrocarbyl” refer to hydrocarbyl in which at least one carbon atom is replaced with a heteroatom. Similarly, “substituted hydrocarbylene” refers to hydrocarbylene substituted with one or more substituent groups, and the terms “heteroatom-containing hydrocarbylene” and heterohydrocarbylene” refer to hydrocarbylene in which at least one carbon atom is replaced with a heteroatom. Unless otherwise indicated, the term “hydrocarbyl” and “hydrocarbylene” are to be interpreted as including substituted and/or heteroatom-containing hydrocarbyl and hydrocarbylene moieties, respectively.

The term “heteroatom-containing” as in a “heteroatom-containing hydrocarbyl group” refers to a hydrocarbon molecule or a hydrocarbyl molecular fragment in which one or more carbon atoms is replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.”

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include, without limitation: functional groups such as halo, hydroxyl, sulfhydryl, C₁-C₂₄ alkoxy, C₂-C₂₄ alkenyloxy, C₂-C₂₄ alkynyloxy, C₅-C₂₄ aryloxy, acyl (including C₂-C₂₄ alkylcarbonyl (—CO-alkyl) and C₆-C₂₄ arylcarbonyl (—CO-aryl)), acyloxy (—O-acyl), C₂-C₂₄ alkoxycarbonyl (—(CO)—O-alkyl), C₆-C₂₄ aryloxycarbonyl (—(CO)—O-aryl), halocarbonyl (—CO)-X where X is halo), C₂-C₂₄ alkylcarbonato (—O—(CO)—O-alkyl), C₆-C₂₄ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-(C₆-C₂₄ aryl)-substituted carbamoyl (—(CO)—NH-aryl), di-(C₆-C₂₄ aryl)-substituted carbamoyl (—(CO)—N(aryl)₂), di-N—(C₁-C₂₄ alkyl), N—(C₆-C₂₄ aryl)-substituted carbamoyl, thiocarbamoyl (—(CS)—NH₂), carbamido (—NH—(CO)—NH₂), cyano(—C≡N), isocyano (—N⁺≡C⁻), cyanato (—O—C≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido (—NH—(CO)-alkyl), C₆-C₂₄ arylamido (—NH—(CO)-aryl), imino (—CR═NH where R=hydrogen, C₁-C₂₄ alkyl, C₅-C₂₄ aryl, C₆-C₂₄ alkaryl, C₆-C₂₄ aralkyl, etc.), alkylimino (—CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR═N(aryl), where R=hydrogen, C₁-C₂₄ alkyl, C₅-C₂₄ aryl, C₆-C₂₄ alkaryl, C₆-C₂₄ aralkyl, etc.), nitro (—NO₂), nitroso (—NO), sulfo (—SO₂—OH), sulfonato (—SO₂—O), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₅-C₂₄ arylsulfonyl (—SO₂-aryl), boryl (—BH₂), borono (—B(OH)₂), boronato (—B(OR)₂ where R is alkyl or other hydrocarbyl), phosphono (—P(O)(OH)₂), phosphonato (—P(O)(O⁻)₂), phosphinato (—P(O)(O⁻)), phospho (—PO₂), and phosphino (—PH₂); and the hydrocarbyl moieties C₁-C₂₄ alkyl (preferably C₁-C₁₂ alkyl, more preferably C₁-C₆ alkyl), C₂-C₂₄ alkenyl (preferably C₂-C₁₃ alkenyl, more preferably C₂-C₆ alkenyl), C₂-C₂₄ alkynyl (preferably C₂-C₁₂ alkynyl, more preferably C₂-C₆ alkynyl), C₅-C₂₄ aryl (preferably C₅-C₁₄ aryl), C₆-C₂₄ alkaryl (preferably C₆-C₁₆ alkaryl), and C₆-C₂₄ aralkyl (preferably C₆-C₁₆ aralkyl).

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.”

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

In the molecular structures herein, the use of bold and dashed lines to denote particular conformation of groups follows the IUPAC convention. A bond indicated by a broken line indicates that the group in question is below the general plane of the molecule as drawn (the “α” configuration), and a bond indicated by a bold line indicates that the group at the position in question is above the general plane of the molecule as drawn (the “β” configuration).

II. Reactants:

The propargylic substrate that is catalytically transformed using the method of the invention is an alkyne substituted at the propargylic position with a leaving group that can be displaced by an incoming nucleophile in a nucleophilic substitution reaction. The alkyne reactant has the structure of formula (I)

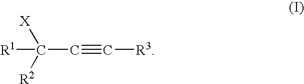

In formula (I), X is the leaving group, and may be, for example, —OH, —OR⁴, —SH, or —SR⁵, wherein R⁴ and R⁵ are selected from C₁-C₂₄ hydrocarbyl, substituted C₁-C₂₄ hydrocarbyl, heteroatom-containing C₁-C₂₄ hydrocarbyl, substituted heteroatom-containing C₁-C₂₄ hydrocarbyl, and activating groups that promote the displacement of X by the nucleophilic reactant. One significant advantage of the invention, however, is that such activating groups are not necessarily, and that a hydroxyl group or an alkoxy moiety per se can serve as the displaceable leaving group. Accordingly, preferred X substituents are selected from —OH and —OR⁴, wherein R⁴ is C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, or substituted C₅-C₁₄ heteroaryl. Optimally, X is —OH.

R¹ is selected from hydrogen, C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl, and is preferably hydrogen or lower hydrocarbyl.

R² is selected from hydrogen, C₁-C₂₄ hydrocarbyl, substituted C₁-C₂₄ hydrocarbyl, heteroatom-containing C₁-C₂₄ hydrocarbyl, substituted heteroatom-containing C₁-C₂₄ hydrocarbyl, and functional groups, and R³ is selected from hydrogen, silyl, C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl. In a preferred embodiment, R² and R³ are independently selected from hydrogen, C₁-C₂₄ alkyl, C₁-C₂₄ heteroalkyl, C₅-C₂₄ aryl, C₅-C₂₄ heteroaryl, C₆-C₂₄ alkaryl, C₆-C₂₄ heteroalkaryl, C₆-C₂₄ aralkyl, and C₆-C₂₄ heteroaralkyl, any of which, with the exception of hydrogen, may be substituted. In a still more preferred embodiment, R² and R³ are independently selected from hydrogen, C₁-C₁₂ alkyl, C₁-C₁₂ heteroalkyl, C₅-C₁₄ aryl, C₅-C₁₄ heteroaryl, C₆-C₁₆ alkaryl, C₆-C₁₆ heteroalkaryl, C₆-C₁₆ aralkyl, and C₆-C₁₆ heteroaralkyl, any of which, again, with the exception of hydrogen, may be substituted. R² and R³ thus include optionally substituted lower alkyl and optionally substituted phenyl.

The nucleophilic reactant serves to displace the leaving group X in a substitution reaction. Any nucleophilic reactant may be used that serves this purpose, and the choice of reactant will depend on the particular leaving group. Generally, however, it will be appreciated that suitable nucleophilic reactants are compounds comprising a nucleophilic group selected from hydroxyl, hydrocarbyloxy, primary amino, secondary amino, silyl, alkenyl, aryl, and heteroaryl, any of which, with the exception of hydroxyl, may be further substituted and/or heteroatom-containing.

Accordingly, nucleophilic reactants include, but are not limited to, the following:

R⁶—OH, wherein R⁶ is selected from selected from C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl;

R⁷—O—R⁸, wherein R⁷ and R³ are defined as for R⁶, and further wherein R⁷ and R⁸ may be linked to form a cyclic ether;

R⁹—NH—R¹⁰, wherein R⁹ is selected from selected from C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl, and R¹⁰ is selected from hydrogen, C₁-C₂₄ hydrocarbyl, substituted C₁-C₂₄ hydrocarbyl, heteroatom-containing C₁-C₂₄ hydrocarbyl, substituted heteroatom-containing C₁-C₂₄ hydrocarbyl, amine-protecting groups, and functional groups, and further wherein R⁹ and R¹⁰ may be linked to form a cyclic amine;

R¹¹—Si(R¹²R¹³R¹⁴), wherein R¹¹ is hydrogen, cyano, cyanato, azido, or boronato, and R¹², R¹³ and R¹⁴ are independently selected from selected from C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl;

R¹⁵R¹⁶C═CR¹⁷R¹⁸ wherein R¹⁵ is an electron-donating substituent, and R¹⁶, R¹⁷, and R¹⁸ are selected from hydrogen, C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl, and further wherein any two of R¹⁶, R¹⁷, and R¹⁸ may be linked to form a cyclic olefin; and

Ar(R¹⁹)ₘ wherein Ar is C₅-C₂₄ aryl, substituted C₅-C₂₄ aryl, C₅-C₂₄ heteroaryl, or substituted C₅-C₂₄ heteroaryl, R¹⁹ is an electron-donating substituent, and m is at least 1, wherein, when m is 2 or more, the R¹⁹ substituents may be the same or different.

Any substituents may be present on R⁶—OH, R⁷—O—R⁸, and R⁹—NH—R¹⁰, and R¹¹—Si(R¹²R¹³R¹⁴), so long as they do not interfere with the desired nucleophilic substitution. Electron-withdrawing substituents will tend to increase the rate at which certain nucleophilic compounds, e.g., alcohols, ethers, and amines, react as nucleophiles, as will be appreciated by those of ordinary skill in the art, but the invention is not limited in this regard. As noted above, both aromatic nucleophiles herein and olefinic nucleophiles in which the double bond acts as the nucleophilic group are substituted with electron-donating substituents. Electron-donating groups include, for example, alkyl, alkoxy, aryl, aryloxy, alkaryl, silyl (e.g., trialkylsilyl), alkylamino, amino, alkylthio, and acyloxy, while representative electron-withdrawing substituents include halo, cyano, haloalkyl, alkylsulfonyl, arylsulfonyl, alkylcarbonyl, alkoxycarbonyl, and formyl.

More preferred nucleophilic reactants are as follows.

(1) R⁶—OH, wherein R⁶ is selected from C₁-C₁₂ alkyl, substituted C₁-C₁₂ alkyl, C₁-C₁₂ heteroalkyl, substituted C₁-C₁₂ heteroalkyl, C₁-C₁₂ alkenyl, substituted C₁-C₁₂ alkenyl, C₁-C₁₂ heteroalkenyl, substituted C₁-C₁₂ heteroalkenyl, C₅-C₁₄ aryl, substituted C₁-C₁₂ alkenyl, C_{C}₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl. R⁶ is not generally substituted with a hydroxyl groups, i.e., these nucleophilic reactants are typically monohydric alcohols. Specific monohydric alcohols suitable as nucleophilic reactants herein include, by way of example and not limitation, methanol, ethanol, 1-chloroethanol, 1-bromoethanol, 1-methoxyethanol, 1-ethoxyethanol, 1-(n-propoxy)-ethanol, 1-isopropoxyethanol, 1-(n-butoxy)-ethanol, 2-chloroethanol, 2-bromoethanol, 2-methoxyethanol, 2-ethoxyethanol, 2-(n-propoxy)-ethanol, 2-isopropoxyethanol, 2-(n-butoxy)-ethanol, propan-1-ol, 1-chloro-propan-1-ol, 1-bromo-propan-1-ol, 1-methoxy-propan-1-ol, 1-ethoxy-propan-1-ol, 1-(n-propoxy)-propan-1-ol, 1-(isopropoxy)propan-1-ol, 1-(n-butoxy)-propan-1-ol, 2-chloro-propan-1-ol, 2-bromo-propan-1-ol, 2-methoxypropan-1-ol, 2-ethoxy-propan-1-ol, 2-(n-propoxy)-propan-1-ol, 2-(isopropoxy)-propan-1-ol, 2-(n-butoxy)-propan-1-ol, 3-chloro-propan-1-ol, 3-bromo-propan-1-ol, 3-methoxy-propan-1-ol, 3-ethoxy-propan-1-ol, 3-(n-propoxy)-propan-1-ol, 3-(isopropoxy)-propan-1-ol, 3-(n-butoxy)-propan-1-ol, 1-chloro-propan-2-ol, 1-bromo-propan-2-ol, 1-methoxy-propan-2-ol, 1-ethoxy-propan-2-ol, 1-(n-propoxy)-propan-2-ol, 1-(isopropoxy)-propan-2-ol, 1-(n-butoxy)-propan-2-ol, 2-chloro-propan-2-ol, 2-bromo-propan-2-ol, 2-methoxy-propan-2-ol, 2-ethoxy-propan-2-ol, 2-(n-propoxy)-propan-2-ol, 2-(isopropoxy)-propan-2-ol, 2-(n-butoxy)-propan-2-ol, prop-2-en-1-ol, 1-chloro-prop-2-en-1-ol, 2-chloro-prop-2-en-1-ol, 3-chloro-prop-2-en-1-ol, 1-methoxy-prop-2-en-ol, but-3-en-1-ol, 1-chloro-but-3-en-1-ol, 2-chloro-but-3-en-1-ol, 3-chloro-but-3-en-1-ol, 4-chloro-but-3-en-1-ol, 1-methoxy-but-3-en-1-ol, 2-methoxy-but-3-en-1-ol, 3-methoxy-but-3-en-1-ol, 4-methoxy-but-3-en-1-ol, phenol, phenyl-methanol, 1-phenyl-ethanol, (4-methoxy-phenyl)methanol, (3,5-dimethoxy-phenyl)-methanol, (2-chloro-phenyl)-methanol, (3,5-dichloro-phenyl)methanol, cyclohexyl-methanol, (tetrahydropyran-3-yl)-methanol, (tetrahydropyran-2-yl)-methanol, (2,2,7,7-tetramethyl-tetrahydro-bis[1,3]dioxolo[4,5-b;4′,5′-d]pyran-5-yl)-methanol, and 3-hydroxy-2-methyl-propionic acid methyl ester.

(2) R⁷—O—R⁸, wherein R⁷ and R⁸ are defined as for R⁶, and further wherein R⁷ and R/⁸ may be linked to form a cyclic ether. Specific examples of ethers that are suitable nucleophilic reactants herein include, without limitation, dimethyl ether, ethyl methyl ether, methyl n-propyl ether, isopropyl methyl ether, methyl n-butyl ether, methyl n-pentyl ether, methyl n-hexyl ether, methyl n-heptyl ether, diethyl ether, ethyl n-hexyl ether, 1-chloro-2-ethoxyethane, 2-chloro-2-ethoxyethane, 1-chloro-1-methoxypropane, 1,1,1-trichloro-2-methoxy-propane, tetrahydropyran, 4-chloro-tetrahydropyran, 3,5-dichloro-tetrahydropyran, 4-methoxy-tetrahydropyran, tetrahydrofuran, 2,3-dichloro-tetrahydrofuran, 2,3-dimethoxy-tetrahydrofuran, N-methyl morpholine, N-ethyl morpholine, N-phenyl morpholine, 4-methoxy-octan-3-one, and 2-methoxybutyric acid methyl ester.

(3) R⁹—NH—R¹⁰, wherein R⁹ is selected from C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl, and R¹⁰ is selected from hydrogen, C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, substituted C₆-C₁₆ heteroaralkyl, C₂-C₁₂ alkoxycarbonyl, substituted C₂-C₁₂ alkoxycarbonyl, and C₆-C₁₄ aryloxycarbonyl, and further wherein R⁹ and R¹⁰ may be linked to form a five-or six-membered N-heterocycle optionally substituted and/or containing additional heteroatoms. Specific examples of amines and other nitrogenous compounds that are suitable as nucleophilic reactants herein, include, without limitation, methylamine, ethylamine, s-butylamine, isopentyl amine, n-hexylamine, cyclohexylamine, dodecylamine, benzylamine, 4-chlorobenzylamine, 4-bromobenzylamine, 3,5-methoxybenzylamine, phenethylamine, dimethylamine, diethylamine, diisopropylamine, ethyl methyl amine, n-butyl methyl amine, piperidine, pyrrolidine, p-toluenesulfonamide, N-methyl-p-toluenesulfonamide, N-methyl ethylcarbamate, N-methyl-n-propylcarbamate, N-methyl cyclohexylarbamate, N-(4-chlorophenyl)methylcarbamate, N-(4-chlorophenyl)ethylcarbamate, N-(3,5-dichlorophenyl)ethylcarbamate, N-(3,5-dichlorophenyl)cyclohexylcarbamate, etc.

(4) H—Si(R¹²R¹³R¹⁴), wherein R¹², R¹³ and R¹⁴ are independently selected from selected from C₁-C₁₂ alkyl and C₅-C₁₄ aryl. Representative such nucleophilic compounds include trimethylsilane, triethylsilane, methyl diethylsilane, dimethyl phenyl silane, methyl diphenyl silane, triphenyl silane, etc.

(5) R¹⁵R¹⁶C═CR¹⁷R¹⁸ wherein R¹⁵ is an electron-donating substituent, and R¹⁶, R¹⁷, and R¹⁸ are selected from hydrogen, C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl, and further wherein any two of R¹⁶, R¹⁷, and R¹⁸ may be linked to form a five- or six-membered cyclic olefin. Examples of these olefinic nucleophiles include, without limitation, 2-methoxy-propene, 2-ethoxy-propene, 2-phenoxy-propene, 3-methoxy-propene, 3-ethoxy-propene, 3-phenoxy-propene, 3-(4-methoxyphenoxy)-propene, 3-(3,5-dimethoxyphenoxy)-propene, 1-methoxy-3-methyl-but-2-ene, 2-methoxy-3-methyl-but-2-ene, (3-methoxy-propenyl)-benzene, 1,3-dimethyl-5-vinyl-benzene, 4-methoxy-5-vinyl-benzene, 1,3-dimethoxy-5-vinyl-benzene, vinyloxymethyl benzene, 1-isopropenyloxymethyl-4-methoxybenzene, 1-isopropenyloxy-cyclohexane, 1-isopropenyloxy-4-methoxy-cyclohexane, (2-methoxy-ethylidene)-cycloheptane, allyl-trimethyl-silane, but-2-enyl-trimethyl-silane, allyloxy-trimethyl-silane, but-2-enyloxy-trimethyl-silane, cyclohex-1-enylmethyl-trimethyl-silane, (cyclohex-1-enylmethoxy)-trimethyl-silane, cyclohex-2-enylmethyl-trimethyl-silane, (cyclohex-2-enylmethoxy)-trimethyl-silane, (cyclohex-1-enylmethoxy)-trimethyl-silane, (cyclohex-2-enyloxy)-trimethyl-silane, etc.

(6) Ar(R¹⁹)ₘ wherein Ar is C₅-C₁₄ aryl, substituted C₅-C₁₄ aryl, C₅-C₁₄ heteroaryl, or substituted C₅-C₁₄ heteroaryl, R¹⁹ is an electron-donating substituent, and m is 1 or 2, wherein, when m is 2, the R¹⁹ substituents may be the same or different. Any two substituents on Ar may also be linked to form an additional cyclic group, which may or may not be aromatic. Such nucleophiles include, for example, methoxybenzene, ethoxybenzene, 4-methoxy-toluene, 4-ethoxy-toluene, 1-benzyloxy-toluene, 2,4-dimethoxy-benzene, 2,4-dimethoxy-toluene, 2,4-diethoxy-benzene, 2,4-dimthoxy-toluene, 4-methylanisole, benzo[1,3]dioxol-5-ol, 2,2-dimethyl-benzo[1,3]dioxol-5-ol, 3,4-dimethoxyphenol, trimethyl-m-tolyl-silane, and 5-allyl-benzo[1,3]dioxole.

III. Catalysts:

The catalysts used in conjunction with the method of the invention are rhenium (V) oxo complexes having at least one electron donor ligand and at least two anionic ligands, under reaction conditions effective to provide for nucleophilic displacement of the leaving group. Exemplary transition metal complexes for use in conjunction with the methods of the invention have the structure of formula (II)

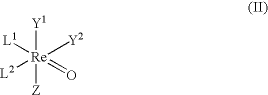

wherein the various substituents are as follows:

L¹ and L² are neutral electron donor ligands, and may be the same or different. L¹ and L² may individually represent monodentate ligands, or they may be taken together to form a single bidentate ligand in which at least one of the coordinating heteroatoms is other than N. Examples of suitable monodentate ligands include, without limitation, phosphine, sulfonated phosphine, phosphite, phosphinite, phosphonite, arsine, stibine, ether (including cyclic ethers), amine, amide, imine, sulfoxide, carboxyl, nitrosyl, pyridine, substituted pyridine (e.g., halogenated pyridine), imidazole, substituted imidazole (e.g., halogenated imidazole), pyrazine (e.g., substituted pyrazine), and thioether. In more preferred embodiments, L¹ and L² are independently selected from phosphines of the formula P(R²⁰)₃, where each R²⁰ is independently monocyclic aryl, C₁-C₁₀ alkyl, substituted C₁-C₁₀ alkyl, substituted monocyclic aryl, or C₁-C₁₀ alkyl. In still more preferred embodiments, L¹ and L² are independently selected from tricyclohexylphosphine, tricyclopentylphosphine, triphenylphosphine, tri(m-tolyl)phosphine, tri (p-tolyl)phosphine, and cyclohexyldiphenylphosphine. Optimally, the monodentate ligands are tricyclohexylphosphine, tricyclopentylphosphine, or triphenylphosphine. Bidentate ligands are described infra.

Y² and Y² are anionic ligands, and may be the same or different. In preferred embodiments, Y¹ and Y² are independently selected from hydride, halide, C₁-C₂₄ alkyl, C₅-C₂₄ aryl, C₁-C₂₄ alkoxy, C₅-C₂₄ aryloxy, C₃-C₂₄ alkyldiketonate, C₅-C₂₄ aryldiketonate, C₂-C₂₄ alkoxycarbonyl, C₅-C₂₄ aryloxycarbonyl, C₂-C₂₄ acyl, C₁-C₂₄ alkylsulfonato, C₅-C₂₄ arylsulfonato, C₁-C₂₄ alkylsulfanyl, C₅-C₂₄ arylsulfanyl, C₁-C₂₄ alkylsulfinyl, or C₅-C₂₄ arylsulfinyl, any of which, with the exception of hydride and halide, are optionally further substituted with one or more groups selected from halide, C₁-C₆ alkyl, C₁-C₆ alkoxy, and phenyl. In more preferred embodiments, Y¹ and Y² are halide, benzoate, C₂-C₆ acyl, C₂-C₆ alkoxycarbonyl, C₁-C₆ alkyl, phenoxy, C₁-C₆ alkoxy, C₁-C₆ alkylsulfanyl, aryl, or C₁-C₆ alkylsulfonyl. In even more preferred embodiments, Y¹ and Y² are each halide, CF₃CO₂, CH₃CO₂, CFH₂CO₂, (CH₃)₃CO, (CF₃)₂(CH₃)CO, (CF₃)(CH₃)₂CO, phenoxy, methoxy, ethoxy, tosylate, mesylate, or trifluoromethanesulfonate. In the most preferred embodiments, Y¹ and Y² are lower alkoxy or halide, e.g., methoxy, ethoxy, chloride or iodide.

Z is a ligand that may be a neutral electron donor ligand, and thus defined as for L¹ and L², or it may be an anionic ligand, and thus defined as for Y¹ and Y².

Therefore, preferred catalysts of formula (II) are those wherein:

L¹ and L² are independently selected from phosphines of the formula P(R²⁰)₃, where each R²⁰ is independently monocyclic aryl, C₁-C₁₀ alkyl, substituted C₁-C₁₀ alkyl, substituted monocyclic aryl, or C₁-C₁₀ alkyl; and

Y¹ and Y² are selected from halide and lower alkoxy, wherein Z is selected from tricyclohexylphosphine, tricyclopentylphosphine, triphenylphosphine, tri(m-tolyl)phosphine, tri (p-tolyl)phosphine, and cyclohexyldiphenylphosphine.

More preferred catalysts of formula (II) are those wherein L¹ and L² are selected from tricyclohexylphosphine, tricyclopentylphosphine, triphenylphosphine, tri(m-tolyl)phosphine, tri (p-tolyl)phosphine, and cyclohexyldiphenylphosphine; and Y¹ and Y² are halide, wherein Z is tricyclohexylphosphine, tricyclopentylphosphine, triphenylphosphine, halide or lower alkoxy.

In another embodiment, L¹ and L² together form a bidentate ligand in which at least one coordinating heteroatom is other than N. One group of such complexes is represented by the structure of formula (III)

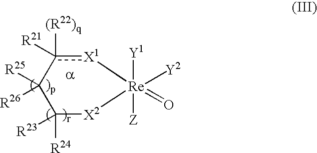

wherein:

α is an optional double bond;

p is zero, 1, or 2;

q is zero when α is present, and q is 1 when α is absent;

r is zero or 1;

and the various substituents are as follows:

X¹ is selected from O, P(R²⁷R²⁸), and NR²⁹ wherein R²⁷, R²⁸, and R²⁹ are independently selected from C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl, and when X¹ is N, then α is present.

X² is selected from O and P(R^{27A}R^{28A}), wherein R^{27A} and R^{28A} are defined as for R²⁷ and R²⁸, respectively.

R²¹, R²², R²³, and R²⁴ are independently selected from hydrogen, C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl.

R²⁵ and R²⁶ are independently selected from hydrogen, C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, substituted C₆-C₁₆ heteroaralkyl, and functional groups.

Additionally, any two or more of R², R²², R²³, R²⁴; R²⁵, R²⁶ R²⁷, R²⁸, and R²⁹ may be linked to form a cyclic group.

Examples of such catalysts are those wherein X¹ and X² are O, p is 1, q is zero, r is 1, and R²⁴ and R²⁶ are hydrogen, such that the complex contains a substituted or unsubstituted acetylacetonate (acac) ligand and has the structure of formula (IV)

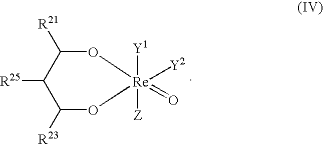

Preferred such catalysts include those wherein R²¹, R²³, and R²⁵ are hydrogen (such that the bidentate ligand shown is acetylacetonate), Y¹ and Y² are halide, and Z is halide, tricyclohexylphosphine, tricyclopentylphosphine, or triphenylphosphine.

Additional examples of catalysts having the structure of formula (III) are those wherein a is present, X¹ is NR²⁹, X² is O, p is 1, q is zero, r is 1, R²⁴ and R²⁶ are hydrogen, such that the complex has the structure of formula (V)

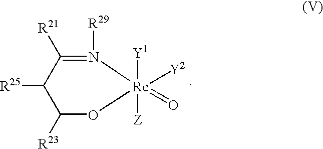

In one specific embodiment of the complex of formula (V), R²³ and R²⁵ are linked to form a phenyl group and R²¹ and R²⁹ are linked to form a 4,5-dioxazole ring, such that the complex has the structure of formula (VI)

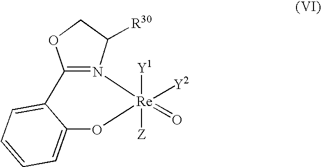

wherein R³⁰ is selected from hydrogen, C₁-C₁₂ alkyl, phenyl, and benzyl.

Still additional complexes of formula (III) are those wherein X¹ is PR²⁷R²⁸ and X² is PR^{27A}R^{28A}, such that the complex contains a bisphosphine ligand. Preferred complexes within this group are those wherein a is absent, q is 1 and R²⁷, R²⁸, R^{27A}, and R^{28A} are aryl, more preferably phenyl. In the latter case, it will be appreciated that when r is zero, p is zero, and R²¹ and R²² are hydrogen, that the complex is (dppm)ReO(Y¹Y²Z). When r is 1, p is zero, and R²¹, R²², R²³, and R²⁴ are hydrogen, the complex is then (dppe)ReO(Y¹Y²Z), while when r is 1, p is 1, and R²¹, R²², R²³, R²⁴, R²⁵, and R²⁶ are hydrogen, then the complex is (dppp)ReO(Y¹Y²Z).

Other complexes containing a bisphosphine ligand and suitable as catalysts herein have the structure of formula (VII)

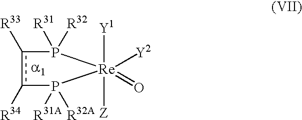

wherein α₁ is an optional double bond, R³¹, R³², R³¹A, R³²A, R³³, and R³⁴ are independently selected from C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl, and wherein any two or more of R³¹, R³², R^{31A}, R^{32A}, R³³, and R³⁴ may be taken together to form a cyclic group, and Y¹, Y², and Z are as defined previously. Optimally, α₁ is present, R³³ and R³⁴ taken together are aryl, e.g., phenyl or naphthalenyl, and R³¹, R³², R^{31A}, and R^{32A} are aryl, preferably phenyl.

Still other rhenium (V) complexes suitable as catalysts herein and containing a bisphosphine ligand are those having the structure of formula (VIII)

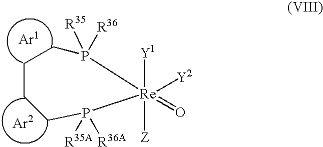

in which:

Ar¹ and Ar² are independently selected from C₅-C₂₄ aryl, substituted C₅-C₂₄ aryl, C₅-C₂₄ heteroaryl, and substituted C₅-C₂₄ heteroaryl;

R³⁵, R³⁶, R^{35A}, and R^{36A} are independently selected from C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl, and wherein any two or more of R³¹, R³², R^{31A}, R^{32A}, R³³, and R³⁴ may be taken together to form a cyclic group; and Y¹, Y², and Z are as defined previously.

Preferably, R³⁵, R³⁶, R^{35A}, and R^{36A} are aryl, and, more preferably, are phenyl. Exemplary Ar¹ and Ar² moieties are phenyl and naphthalenyl.

Additional complexes suitable as reaction catalysts herein have the structure of formula (IX)

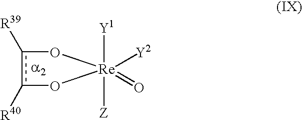

wherein:

α₂ is an optional double bond;

R³⁹ and R⁴⁰ are independently selected from C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl, and wherein R³⁹ and R⁴⁰ may be taken together to form a cyclic group; and Y¹, Y², and Z are as defined previously.

Preferred catalysts within those of formula (IX) are wherein α₂ is present and R³⁹ and R⁴⁰ taken together form a phenyl ring.

IV. Reaction Conditions:

In a preferred embodiment, the method of the invention is carried out using an excess of the nucleophilic reactant, i.e., the molar ratio of the nucleophilic reactant to the alkyne reactant is greater than 1:1. Preferably, the molar ratio of the nucleophilic reactant to the alkyne reactant is in the range of about 1.5:1 to about 3:1. The reaction is conducted in a polar aprotic solvent (e.g., acetonitrile, nitromethane, tetrahydrofuran, chlorobenzene, and the like) at a temperature in the range of about 20° C. to about 80° C., and the amount of catalyst used is on the order of 5 mole % or less, preferably on the order of 1 mole % or less, and even as low as 0.1 mole %. In order to ensure that the catalyst can be recovered, the reaction is preferably carried out at ambient temperature, typically in the range of about 20° C. to about 25° C. Catalyst recovery is readily accomplished by addition of a nonpolar solvent to the reaction mixture in an amount to result in precipitation of the catalyst, which can then be removed using conventional techniques such as filtration or solvent evaporation.

The metal complex of formula (III) and other complexes herein that are employed as catalysts may be used with a co-catalyst, although a co-catalyst is not required. Suitable co-catalysts are those composed of a cationic component capable of abstracting an anionic ligand from the metal complex and an anion that does not coordinate to the rhenium center. When co-catalysts are used, then, it will be appreciated that the metal complex is in the form of a cation in association with the anion of the co-catalyst. Preferred co-catalysts contain anions that are sterically bulky, so that the negative charge borne by the ion is delocalized. Weakly coordinating bulky anions are known to those of ordinary skill in the art, and include, by way of example and not limitation, fluorohydrocarbylborate ions, trifluoromethanesulfonate, BF₄⁻, Ph₄B⁻ (Ph=phenyl), p-toluenesulfonate, SbF₆⁻, and PF₆⁻. Particularly preferred such anions are the fluorohydrocarbylborate ions, e.g., tetrakis[3,5-bis(trifluoromethyl)phenyl]borate (BAF⁻), tetra(pentafluorophenyl)borate, H⁺(OCH₂CH₃)₂[(bis-3,5-trifluoromethyl)phenyl]borate, and trityltetra(pentafluorophenyl)borate.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, and publications mentioned herein are hereby incorporated by reference in their entireties.

Experimental:

Unless otherwise noted all commercial materials were used without further purification. Reactions were carried out in two dram vials fitted with threaded caps. ACS grade acetonitrile and methanol were obtained from FM science. 2-propanol was obtained from Fisher Scientific. 3-chloro-1-propanol, sec-phenethyl alcohol, 2-methoxyethanol, and isopropanol were obtained from Aldrich Chemical Company. 3-buten-1-ol was obtained from Fluka Chemika. mer-[ReOCl₃(dppm)] was prepared according to literature procedures (Chat et al. (1962) J. Chem. Soc., at 4019; Rossi et al. (1993) Inorg. Chim. Acta. 204: 63). Optically pure 1-phenyl-2-heptyn-1-ol was obtained according to the procedure described in Midland et al. (1984) Tetrahedron 40:1371, and enantiomeric purity was determined by chiral HPLC analysis, Chiralcel OD column, 95:5 hexanes:2-propanol, 1 mL/min; retention times 9.09 and 13.58 min. Analytical thin-layer chromatography (TLC) of reaction mixtures was preformed on Merck silica gel 60 F₂₅₄ TLC plates. Chromatography was carried out on ICN SiliTech 32-63 D 60 Å silica gel. ¹H and ¹³C NMR spectra were recorded with Bruker AMX-300 and AMX-400 spectrometers and referenced to CDCl₃ unless otherwise noted. Mass spectral and CHN data were obtained via the Micro-Mass/Analytical Facility operated by the College of Chemistry, University of California, Berkeley.

General procedure for propargylic etherification reactions catalyzed by bis(triphenylphosphine) oxorhenium (V) trichloride (mer-[ReOCl₃(dppm)]): A 100 mg sample of propargyl alcohol was dissolved in 0.5 mL of MeCN in a two dram vial. Three equivalents of alcohol nucleophile and 1 mol % catalyst were added. The vial was capped and placed in a 65° oil bath. Reactions were maintained at the temperature indicated until complete as judged by TLC analysis of the reaction mixture. Crude reaction mixtures were loaded onto a silica gel column and purified by chromatography.

Determination of Chirality Transfer for rhenium-catalyzed propargylation: The substitution reaction was carried out in the manner described above using 1-phenyl-2-heptyn-1-ol of 86% optical purity. The enantiomeric excess of the product was determined by chiral HPLC analysis, Chiralcel OD column, 98:2 hexanes:2-propanol, 0.5 mL/min; retention times 11.18 and 13.54 min.

### EXAMPLE 1

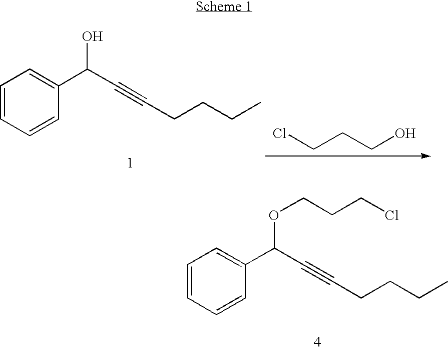

A variety of metal-oxo complexes were examined for their capability of selectively converting propargyl alcohol 1 to propargyl ether 4, as illustrated in Scheme 1. The complexes included V(O)(acac)₂, [Mo₂O₇(BINOL)₂](NBu₄)₂, MoO₂(acac)₂, (PPh₃)₂Re(catechol)Cl, and (dppm)ReOCl₃. Each reaction was carried out by admixing 3.0 equivalents of the nucleophilic alcohol with the alkyne in MeCN (the reaction mixture was 1 M with respect to the alkyne in the solvent), and 5 mole % catalyst. The relative quantities of the products obtained were determined by ¹H NMR of the crude reaction mixture, and are indicated in Table 1. As may be seen, the vanadium-oxo complex primarily resulted in oxidation of the propargylic hydroxyl moiety to the corresponding ketone 2

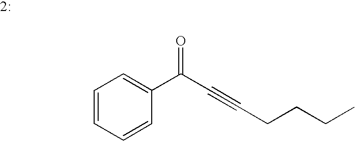

MoO₂(acac)₂ was found to be a somewhat effective catalyst for the substitution reaction with a 1° alcohol nucleophile to provide the desired ether 4 (entry 3), but conversion to the enone 3

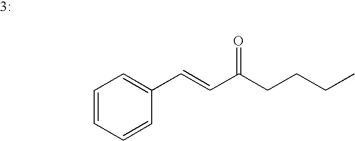

dominated when the nucleophile became more hindered. The rhenium(V)-oxo complex bearing the bidentate phosphine ligand dppm proved to be the most effective catalyst for the desired transformation (entry 5). Furthermore, substitution proceeded smoothly without exclusion of moisture or air from the reaction mixture. The procedure led to the discovery that the catalyst loading could be decreased to 1 mol % without a significant impact on yield or reaction time.

Table 1. Selectivity of Metal-Oxo Catalysts for Propargyl Etherfication

TABLE I
Selectivity of Metal-Oxo Catalysts for Propargyl Etherfication

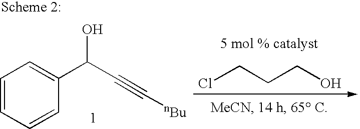

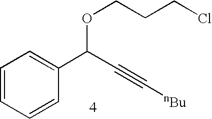

| entry | catalyst | % 2 | % 3 | % 4 |
|---|---|---|---|---|
| 1 | V(O)(acac)₂ | 0 | 29 | 19 |
| 2 | [Mo₂O₇(BINOL)₂]NBu₄)₂ | 0 | 10 | 15 |
| 3 | MoO₂(acac)₂ | 20 | trace | 77 |
| 4 | (catechoi)ReOCl₃ | 75 | 0 | 25 |
| 5 | (dppm)ReOCl₃ | trace | trace | 96 |

With the Optimum conditions having been determined as explained above, the reaction was then carried out according to the general procedure using 1 mole % mer-[ReOCl₃(dppm)] as the catalyst. The product 4 was purified by chromatography on silica gel (9:1 hexane/Et₂O); colorless oil (78%): ¹H NMR (CDCl₃, 300 MHz) δ 7.52-7.48 (m, 2H), 7.39-7.29 (m, 3H), 5.16 (t, 1H, J=2.0 Hz), 3.82-3.77 (m, 1H), 3.67-3.57 (m, 3H), 2.29 (td, 2H, J=7.0, 2.1 Hz), 2.06 (quintet, 2H, J=6.3 Hz), 1.59-1.49 (m, 2H), 1.47-1.39 (m, 2H), 0.92 (t, 3H, J=7.2 Hz) ppm; ¹³C NMR (CDCl₃, 100 MHz) δ 139.2, 128.4, 128.2, 127.3, 88.6, 77.7, 72.0, 64.5, 42.1, 32.8, 30.7, 22.0, 18.5, 13.6 ppm.

The general procedure for propargylic etherification was then repeated with a variety of propargylic alcohol substrates and nucleophilic reactants. The substrates included propargylic alcohols additionally bearing the following substituents at the propargylic position: phenyl (Table 2, entries 1-6; Examples 2-7); heteroaryl (Table 2, entries 7 and 8; Examples 8 and 9); electron rich aryl (Table 2, entries 9-13 and 16; Examples 10-14 and 17), sterically encumbered ortho disubstituted phenyl (Table 2, entry 14; Example 15), acetals (Table 2, entry 15; Example 16), and bromophenyl (Table 2, entry 16; Example 17). The reactants, reaction time, and yield are shown in Table 2:

TABLE 2
Re (V) oxo catalyzed etherification of propargyl alcohols.

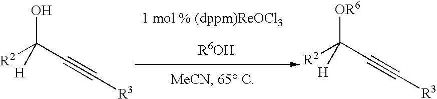

| entry | R² | R³ | R⁶ | time (hr) | yield^{a} |
|---|---|---|---|---|---|
| 1 | 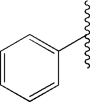 | n-Bu | 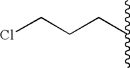 | 8 | 78 |
| 2^{b} | C₆H₅ | 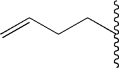 | 14 | 76 | |
| 3^{b} | | SiMe₃ | 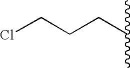 | 8 | 74 |
| 4 | | SiMe₃ | 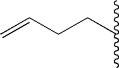 | 8 | 88 |
| 5^{e} | | n-Bu | 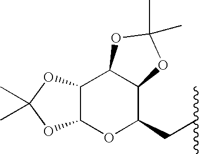 | 10 | 60^{d} |
| 6^{e} | | —(CH₂)₃OH | Me— | 20 | 53 |
| 7 | 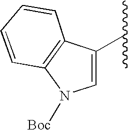 | n-Bu | 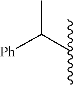 | 5 | 79^{c} |
| 8 | | Me | 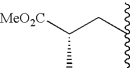 | 7 | 85^{d} |
| 9 | 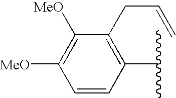 | Me | 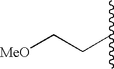 | 2 | 86 |
| 10 | | Me | 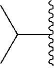 | 2 | 69 |
| 11^{e} | 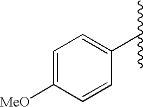 | CO₂Et | 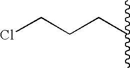 | 10 | 69 |
| 12 | | Me | Me— | 4 | 82 |
| 13^{f} | | Me | Me— | 8 | 77 |
| 14 | 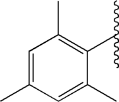 | Me | 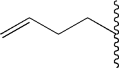 | 5 | 79 |
| 15 | 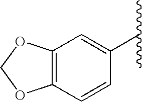 | Me | 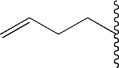 | 2 | 85 |
| 16 | 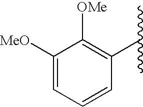 | Me | 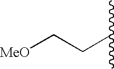 | 7 | 78 |
| 17^{e} | 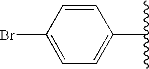 | Me | 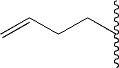 | 10 | 60 |

^{a}Isolated yield after chromatography ^{b}carried out at 80° C. ^{c}obtained as 1:1.6 mixture of diastereomers. ^{d}obtained as 1:1 mixture of diastereomers. ^{e}run with 5 mol % catalyst. ^{f}run with 0.1 mol % catalyst

### EXAMPLE 2 (TABLE 2, ENTRY 2)

The reaction of Table 2, entry 2, was carried out according to the general procedure, and the product was purified by chromatography on silica gel (24:1 hexanes/Et₂O, 1% TEA); yellow oil (76%): ¹H NMR (C₆D₆, 300 MHz) δ 7.60 (app d, 2H, J=7.2 Hz), 7.34-7.31 (m, 2H), 7.16-7.04 (m, 3H), 6.88-6.85 (m, 3H), 5.83-5.69 (m, 1H), 5.28 (s, 1H), 5.01-4.91 (m, 2H), 3.78-3.70 (m, 1H), 3.48-3.41 (m, 1H), 2.31-2.24 (m, 2H) ppm; ¹³C NMR (C₆D₆, 100 MHz) δ 139.3, 135.2, 132.5, 128.4, 128.3, 128.2 (2), 122.8, 116.1, 87.8, 87.4, 72.1, 67.7, 34.2 ppm; CHN and HRMS data were not obtained due to product instability.

### EXAMPLE 3 (TABLE 2, ENTRY 3)

The reaction of Table 2, entry 3, was carried out according to the general procedure, and the product was purified by chromatography on silica gel (10:1 hexanes/Et₂O); colorless oil (74%). ¹H NMR (CDCl₃, 300 MHz) δ 7.53 (m, 2H), 7.38 (m, 3H), 5.20 (s, 1H), 3.81 (m, 1H), 3.66 (m, 3H), 2.08 (app. pentet, 2H, J=6.3 Hz), 0.24 (s, 9H) ppm; ¹³C NMR (CDCl₃, 100 MHz) δ 138.9, 128.2, 127.4, 102.8, 92.7, 72.3, 64.4, 42.1, 32.5, 0.3 ppm; HRMS (EI) calcd for C₁₅H₂₁ClOSi: 280.1050, found: 280.1049; Anal calcd: C, 64.14; H, 7.54. Found: C, 64.96; H, 8.20.

### EXAMPLE 4 (TABLE 2, ENTRY 4)

The reaction of Table 2, entry 4, was carried out according to the general procedure, and the product was purified by chromatography on silica gel (10:1 hexanes/Et₂O); colorless oil (88%). ¹H NMR (CDCl₃, 300 MHz) δ 7.53 (m, 2H), 7.36 (m, 3H), 5.87 (m, 1H), 5.22 (s, 1H), 5.17-5.08 (m, 2H), 3.66 (m, 2H), 2.42 (m, 2H), 0.24 (s, 9H) ppm; ¹³C NMR (CDCl₃, 100 MHz) δ 138.4, 135.1, 128.4, 128.3, 127.5, 116.4, 103.2, 92.5, 71.9, 67.4, 34.1, 29.8, 0.22 ppm; Anal calcd for C₁₆H₂₂OSi: C, 74.36; H, 8.58. Found: C, 74.53, H, 8.89.

### EXAMPLE 5 (TABLE 2, ENTRY 5)

The reaction of Table 2, entry 5, was carried out according to the general procedure, and a 1:1 mixture of diastereomers was obtained. The diastereomeric mixture, inseparable by column chromatography, was purified on silica gel (3:1 hexanes/Et₂O); colorless oil (60%). ¹H NMR (CDCl₃, 300 MHz) The following was observed for the mixture of diastereomers: δ 7.53 (m, 2H), 7.37-7.27 (m, 3H), 5.55 (s, 1H), 5.53 (s, 1H), 5.34 (s, 1H), 5.28 (s, 1H), 4.59 (s, 1H), 4.30 (m, 2H), 4.03 (m, 1H), 3.77, (m, 2H), 2.28 (m, 2H), 1.59-1.26 (m, 4H), 1.55 (s, 3H), 1.44, (s, 3H), 1.34 (s, 3H), 1.33 (s, 3H), 0.92 (t, 3H, J=7.2 Hz) ppm; ¹³C NMR (CDCl₃, 100 MHz) δ 139.2 (2), 128.3, 128.1 (2), 127.5, 109.2, 109.1, 108.5, 96.3, 88.8, 88.6, 77.7, 77.5, 72.0, 71.8, 71.2, 71.0, 70.7(2), 70.6, 67.5, 66.4, 66.2, 66.1, 30.7, 26.1 (2), 26.0, 25.0 (2), 24.5 (2), 22.0, 18.6 (2), 13.6 ppm.

### EXAMPLE 6 (TABLE 2, ENTRY 6)

The reaction of Table 2, entry 6, was carried out according to the general procedure, and the product was purified by chromatography on silica gel (1:1 Et₂O/hexanes); colorless oil (53%); ¹H NMR (CDCl₃, 400 MHz) δ 7.49 (app d, 2H, J=8.0 Hz), 7.39-7.29 (m, 3H), 5.06 (t, 1H, J=2.0 Hz), 3.67 (t, 2H, J=6.2 Hz), 3.40 (s, 3H), 2.34 (td, 2H, J=6.8, 2.0 Hz), 1.73-1.60 (m, 4H) ppm; ¹³C NMR (CDCl₃, 100 MHz) δ 139.0, 128.4,128.3, 127.4, 88.2, 781, 73.2, 62.4, 55.7, 31.9, 24.9, 18.7 ppm.

### EXAMPLE 7 (TABLE 2, ENTRY 7)

The reaction of Table 2, entry 7, was carried out according to the general procedure, and a 1.6:1 mixture of diastereomers was obtained. The diastereomeric mixture, inseparable by column chromatography, was purified on silica gel (10:1 hexanes/Et₂O); light yellow oil (79%). ¹H NMR (CDCl₃, 300 MHz) Major diastereomer: δ 7.74 (s, 1H), 5.14 (s, 1H), 5.06 (q, 1H, J=6.6 Hz), 2.33 (td, 2H, J=6.9, 2.1 Hz), 1.67 (s, 9H), 1.56 (d, 3H, J=6.6 Hz), 0.97 (t, 3H, J=6.9 Hz) ppm; Minor diastereomer: δ 7.87 (d, 1H, J=7.8 Hz), 7.59 (s, 1H), 5.30 (s, 1H), 4.63, (q, 1H, J=6.6 Hz), 2.22 (td, 2H, J=6.9, 2.1 Hz), 1.69 (s, 9H), 1.48 (d, 3H, J=6.6 Hz), 0.88 (t, 3H, J=6.9 Hz) ppm; The following were observed for both diastereomers: δ 8.13 (m, 1H), 7.53-7.19 (m, 6H), 1.84-1.30 (m, 4H) ppm; ¹³C NMR (CDCl₃, 100 MHz) The following were observed for the mixture of diastereomers: δ 149.7, 143.5, 142.9, 135.9, 128.6, 128.5, 128.4, 127.9, 127.5, 127.0, 126.5, 124.6, 124.5, 122.6, 122.5, 120.6, 120.0, 119.9, 119.4, 115.2, 87.3, 86.7, 83.8, 83.6, 78.1, 77.3, 75.2, 74.8, 71.4, 62.6, 61.9, 30.8, 30.6, 28.2 (2), 24.0, 23.8, 22.0 (2), 18.6 (2), 13.7, 13.6 ppm; Anal calcd for C₂₈H₃₃NO₃: C, 77.93; H, 7.71; N, 3.25. Found: C, 78.17; H, 7.98; N, 3.08.

### EXAMPLE 8 (TABLE 2, ENTRY 8)

The reaction of Table 2, entry 4, was carried out according to the general procedure, and a 1:1 mixture of diastereomers was obtained. The inseparable diastereomeric mixture was purified on silica gel (5:1 hexanes/ Et₂O); light yellow oil (85%). ¹H NMR (CDCl₃, 300 MHz) δ 8.11 (m, 1H), 7.96 (m, 2H), 7.34-7.21 (m, 2H), 5.44 (m, 1H), 3.74, (m, 1H), 3.66-3.55 (m, 1H), 3.62 (s, 3H), 2.77 (m, 1H), 1.92 (m, 3H), 1.67 (s, 9H), 1.17 (m, 3H) ppm; ¹³C NMR (CDCl₃, 100 MHz) δ 175.4, 175.1, 149.7, 135.9, 128.6, 128.5, 124.8, 124.6, 122.7, 120.2, 119.1 (2), 115.2 (2), 83.8, 83.0, 75.9 (2), 71.3, 69.2, 68.8, 65.3, 65.1, 51.7, 40.1, 40.0, 28.3, 28.2, 14.2, 3.7 ppm; HRMS (EI) calcd for C₂₂H₂₇NO₅: 385.1889, found: 385.1892.

### EXAMPLE 9 (TABLE 2, ENTRY 9)

The reaction of Table 2, entry 9, was carried out according to the general procedure, and the product was purified by chromatography on silica gel (1:1 hexanes/Et₂O); colorless oil (86%): ¹H NMR (CDCl₃, 300 MHz) δ 7.43 (d, 1H, J=8.7 Hz), 6.82 (d, 1H, J=8.4 Hz), 6.05-5.92 (m, 1H), 5.29 (q, 1H, J=2.1 Hz), 5.02-4.89 (m, 2H), 3.87 (s, 3H), 3.80 (s, 3H), 3.76-3.71 (m, 1H), 3.65-3.60 (m, 1H), 3.58-3.54 (m, 4H), 3.36 (s, 3H), 1.89 (d, 3H, J=2.1 Hz) ppm; ¹³C NMR (CDCl₃, 100 MHz) δ 152.7, 147.3, 137.2, 132.1, 130.3, 123.8, 115.0, 110.4, 83.6, 71.8, 69.1, 67.1, 60.7, 58.9, 55.6, 9.9, 20.3, 3.8 ppm; HRMS (EI) calcd for C₁₈H₂₄O₄ 304.1675 found 304.1678; Anal calcd for C₁₈H₂₄O₄: C, 71.03; H, 7.95. Found: C, 71.46; H, 7.93.

### EXAMPLE 10 (TABLE 2, ENTRY 10)

The reaction of Table 2, entry 10, was carried out according to the general procedure, and the product was purified by chromatography on silica gel (3:1 hexanes/Et₂O); colorless oil (69%): ¹H NMR (CDCl₃, 300 MHz) δ 7.41 (d, 1H, J=8.7 Hz), 6.83 (d, 1H, J=8.7 Hz), 6.05-5.92 (m, 1H), 5.26 (q, 1H, J=2.1 Hz), 5.04-4.91 (m, 2H), 3.91-3.85 (m, 1H), 3.85 (s, 3H), 3.80 (s, 3H), 3.57-3.55 (m, 2H), 1.87 (d, 3H, J=2.1 Hz), 1.19 (app. t, 6H, J=6.3 Hz) ppm; ¹³C NMR (CDCl₃, 100 MHz) δ 152.5, 147.2, 137.2, 131.6, 131.5, 123.5, 115.0, 110.4, 82.5, 78.2, 68.9, 65.7, 60.7, 55.6, 29.8, 22.8, 21.6, 3.8 ppm; HRMS (EI) calcd for C₁₈H₂₄O₃ 288.1725 found 288.1722; Anal calcd for C₁₈H₂₄O₃: C, 74.97; H, 8.39. Found: C, 75.26; H, 8.60.

### EXAMPLE 11 (TABLE 2, ENTRY 11)

The reaction of Table 2, entry 11, was carried out according to the general procedure, and the product was purified by chromatography on silica gel (4:1 hexanes/EtOAc); colorless oil (69%): ¹H NMR (CDCl₃, 300 MHz) δ 7.40 (app. d, 2H, J=6.6 Hz), 6.91 (app. d, 2H, J=6.6 Hz), 5.21 (s, 1H), 4.25 (q, 2H, J=7.1 Hz), 3.81-3.78 (m, 4H), 3.66-3.62 (m, 3H), 2.06 (pentet, 2H, J=6.4 Hz), 1.32 (t, 3H, J=7.2 Hz) ppm; ¹³C NMR (CDCl₃, 100 MHz) δ 160.1, 153.3, 128.8, 114.1, 84.6, 78.8, 71.2, 65.2, 62.2, 55.3, 41.7, 32.6, 14.0 ppm; Anal calcd for C₁₆H₁₉ClO₄: C, 61.84; H, 6.16. Found: C, 61.47; H, 6.27.

### EXAMPLE 12 (TABLE 2, ENTRY 12)

The reaction of Table 2, entry 12, was carried out according to the general procedure, and the product was purified by chromatography on silica gel (9:1 hexanes/Et₂O); colorless oil (82%): ¹H NMR (CDCl₃, 300 MHz) δ 7.42 (app d, 2H, J=8.7 Hz), 6.89 (app d, 2H, J=9.0 Hz), 4.98 (q, 1H, J=2.1 Hz), 3.81 (s, 3H), 3.37 (s, 3H), 1.92 (d, 3H, J=2.4 Hz) ppm; ¹³C NMR (CDCl₃, 100 MHz) δ 159.6, 131.6, 131.3, 128.8, 113.6, 83.7, 72.8, 55.5, 55.3, 3.7 ppm.

### EXAMPLE 13 (TABLE 2, ENTRY 13)

The reaction of Table 2, entry 13, was carried out according to the general procedure, and the product was purified by chromatography on silica gel (24:1 hexanes/Et₂O); colorless oil (79%): ¹H NMR (CDCl₃, 300 MHz) δ 6.83 (s, 2H), 5.87-5.74 (m, 1H), 5.45 (q, 1H, J=2.4 Hz), 5.10-4.99 (m, 2H), 3.64-3.57 (m, 1H), 3.38-3.31 (m, 1H), 2.44 (s, 6H), 2.25 (s, 3H), 1.83 (d, 3H, J=2.1 Hz) ppm; ¹³C NMR (CDCl₃, 100 MHz) δ 137.4, 136.8, 135.3, 132.6, 129.8, 116.3, 82.3, 77.2, 67.7, 67.5, 34.3, 20.9, 20.3, 3.9 ppm; Anal. calcd for C₁₇H₂₂O: C, 84.25; H, 9.15. Found: C, 83.96; H, 9.44.

### EXAMPLE 14 (TABLE 2, ENTRY 14)

The reaction of Table 2, entry 14, was carried out according to the general procedure, and the product was purified by chromatography on silica gel (9:1 hexanes/Et₂O); colorless oil (85%): ¹H NMR (CDCl₃, 300 MHz) δ 7.01 (d, 1H, J=1.5 Hz), 6.96-6.93 (m, 1H), 6.77 (d, 1H, J=7.8 Hz), 5.95 (s, 2H), 5.87 (m, 1H), 5.13-5.01 (m, 3H), 3.66-3.59 (m, 1H), 3.52-3.44 (m, 1H), 2.37 (m, 2H), 1.91 (d, 3H, J=2.1 Hz) ppm; ¹³C NMR (CDCl₃, 100 MHz) δ 147.8, 147.5, 135.2, 133.4, 121.0, 116.4, 107.9, 101.1, 83.6, 77.4, 71.5, 67.2, 63.9, 34.1, 3.8 ppm; HRMS (EI) calcd for C₁₅H₁₆O₃ 244.1096 found 244.1099 (M⁺); Anal calcd for C₁₅H₁₆O₃: C, 73.75; H, 6.60. Found: C, 73.63; H, 6.56.

### EXAMPLE 15 (TABLE 2, ENTRY 15)

The reaction of Table 2, entry 15, carried out according to the general procedure, and the product was purified by chromatography on silica gel (1:1 hexanes/Et₂O); colorless oil (78%): ¹H NMR (CDCl₃, 300 MHz) δ 7.25 (dd, 1H, J=7.6, 1.2 Hz), 7.08 (t, 1H, J=7.9 Hz), 6.87 (dd, 1H, J=8.1, 1.5 Hz), 5.55 (q, 1H, J=2.4 Hz), 3.91-3.76 (m, 2H), 3.87 (s, 3H), 3.86 (s, 3H), 3.65-3.55 (m, 2H), 3.35 (s, 3H), 1.87(d, 3H, J=2.4 Hz) ppm; ¹³C NMR (CDCl₃, 100 MHz) δ 152.5, 146.6, 133.3, 124.1, 120.5, 112.3, 82.9, 71.7, 67.5, 66.22, 61.1, 58.9, 55.8, 3.8 ppm.

### EXAMPLE 16 (TABLE 2, ENTRY 16)

The reaction of Table 2, entry 16, was carried out according to the general procedure, and the product was purified by chromatography on silica gel (5:1 hexanes/Et₂O); colorless oil (60%): ¹H NMR (CDCl₃, 300 MHz) δ 7.47 (m, 2H), 7.37 (m, 2H), 5.82 (m, 1H), 5.13-5.01 (m, 2H), 5.07 (m, 1H), 3.66 (m, 1H), 3.49 (m, 1H), 2.37 (m, 2H), 2.85 (d, 3H, J=2.4 Hz) ppm; ¹³C NMR (CDCl₃, 100 MHz) δ 138.5, 135.1, 131.5, 129.0, 122.1, 116.5, 84.2, 71.2, 71.0, 67.5, 34.1, 3.8 ppm.

### EXAMPLE 17 (TABLE 2, ENTRY 17)

The reaction of Table 2, entry 17, was carried out according to the general procedure, and the product was purified by chromatography on silica gel (24:1 hexanes/Et₂O); colorless oil (53%): ¹H NMR (CDCl₃, 300 MHz) δ 7.46-7.39 (m, 2H), 7.33-7.27 (m, 3H), 3.77 (t, 2H, J=5.8 Hz), 3.68 (t, 2H, J=6.3 Hz), 2.05 (pentet, 2H, J=6.1 Hz), 1.54 (s, 6H) ppm; ¹³C NMR (CDCl₃, 100 MHz) δ 131.7, 128.3, 128.2, 122.9, 91.4, 84.1, 706, 60.5, 42.2, 33.2, 28.4 ppm; HRMS (EI) calcd for C₁₄H₁₇OCl 236.0968 found 236.0968 (M⁺); Anal calcd for C₁₄H₁₇OCl: C, 71.03; H, 7.24. Found: C, 70.90; H, 7.41.

### EXAMPLE 18 (TABLE 2, ENTRY 18)

The reaction of Table 2, entry 18, was carried out according to the general procedure, and the product was purified by chromatography on silica gel (3:1 hexanes/Et₂O); yellow oil (58%): ¹H NMR (CDCl₃, 300 MHz) δ 7.52 (app d, 2H, J=6.6 Hz), 7.34-7.27 (m, 3H), 5.27 (t, 1H, J=1.8 Hz), 3.78-3.63 (m, 2H), 3.60-3.57 (m, 2H), 3.38 (s, 3H), 2.28 (td, 2H, J=7.1, 2.0 Hz), 1.57-1.49 (m, 2H), 1.47-1.38 (m, 2H), 0.91 (t, 3H, J=7.2 Hz) ppm; ¹³C NMR (CDCl₃, 100 MHz) δ 139.1, 128.3, 128.1, 127.5, 88.7, 77.6, 72.0, 71.8, 66.7, 59.0, 30.7, 22.0, 18.6, 13.6 ppm.

Accordingly, as may be deduced from Table 2, variation in the propargylic substituent from alkyl to aryl, trimethylsilyl or ester moiety was well tolerated by the rhenium oxo catalyst, although slightly increased temperatures (entries 1-3) or longer reaction times (entry 11) could be required. Remarkably, substitution of the propargyl alcohol occurred preferentially over conjugate addition to the alkynyl ester (entry 11) and was favored over displacement of other leaving groups on the nucleophile, such as primary alkyl halides (entries 1-3, 11). Primary and secondary alcohols (entries 7, 10) participated as nucleophiles in the reaction without a noticeable difference. In all examples, the reaction regioselectively afforded the propargyl ether, even when competing intramolecular addition of a pendent alcohol would be expected to result in an allene rather than the desired ether.

### EXAMPLE 19

### Propargylic Amination with a Primary Amine

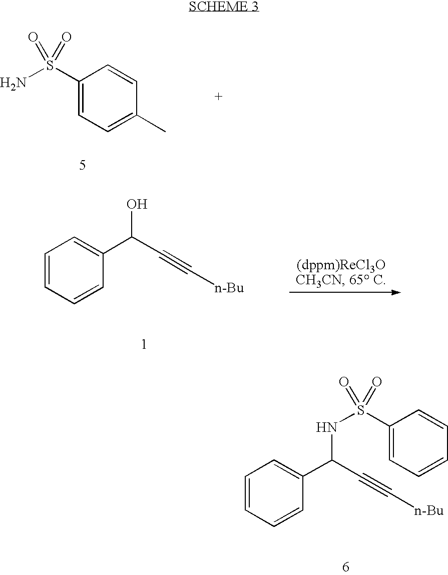

The reaction of Scheme 3 was carried out as follows:

1-Phenyl-2-heptyn-1-ol (1) (100 mg, 0.53 mmol) was added to a 1 dram vial with a threaded cap containing three equivalents of p-toluenesulfonamide (5) (275 mg, 1.59 mmol). To the vial was then added (dppm)ReCl₃O (11 mg, 0.016 mmol, 3 mol %) and ammonium hexafluorophosphate (4.5 mg, 0.027 mmol, 5 mol %) in a solution of 0.5 ml acetonitrile. The resulting green mixture was stirred at 65° C. for three hours. Upon completion, the reaction mixture was chromatographed directly on a silica gel column (2:1 hexanes:ether) to afford 6 as a white solid. ¹H NMR (CDCl₃, 300 MHz) δ 7.76 (m, 2H), 7.45 (m, 2H), 7.32-7.24 (m, 5H), 5.28 (app. d, 1H, J=6.6 Hz), 4.87 (m, 1H), 2.42 (s, 3H), 1.95 (m, 2H), 1.30-1.20 (m, 4H), 0.84 (t, 3H, J=5.4 Hz) ppm; ¹³C NMR (CDCl₃, 100 MHz) δ 143.3, 138.2, 137.6, 129.4, 128.6, 128.2, 127.5, 127.3, 87.5, 76.6, 49.5, 30.3, 21.9, 21.6, 18.3, 13.6 ppm

### EXAMPLE 20

### Propargylic Amination with a Secondary Amine

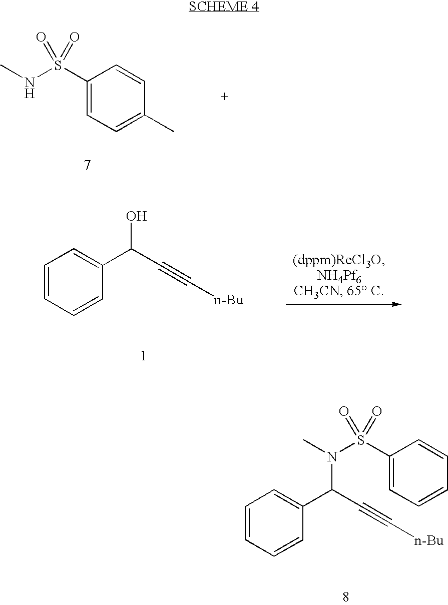

The reaction of Scheme 4 was carried out as follows:

1-Phenyl-2-heptyn-1-ol (1) (100 mg, 0.53 mmol) was added to a 1-dram vial with a threaded cap containing three equivalents of N-methyl p-toluenesulfonamide (7) (295 mg, 1.59 mmol). To the vial was then added (dppm)ReCl₃O (18.5 mg, 0.027 mmol, 5 mol %) and ammonium hexafluorophosphate (4.5 mg, 0.027 mmol, 5 mol %) in a solution of 0.5 ml acetonitrile. The resulting green mixture was stirred at 65° C. for three hours. Upon completion, the reaction mixture was chromatographed directly on a silica gel column (4:1 hexanes:ether) to afford 8 as a white solid (66%). ¹H NMR (CDCl₃, 300 MHz) δ 7.79 (m, 2H), 7.59 (m, 2H), 7.39-7.28 (m, 5H), 6.01 (s, 1H), 2.54 (s, 3H), 2.45 (s, 3H), 1.98 (m, 2H), 1.31 -1.16 (m, 4H), 0.85 (m, 3H) ppm; ¹³C NMR (CDCl₃, 100 MHz) o 143.2, 136.7, 135.1, 129.3, 128.4, 128.1, 128.0, 127.9, 89.4, 73.0, 53.7, 30.5, 29.6, 21.9, 21.6, 18.2, 13.6 ppm.

### EXAMPLE 21

### N-propargylation of a Carbamate

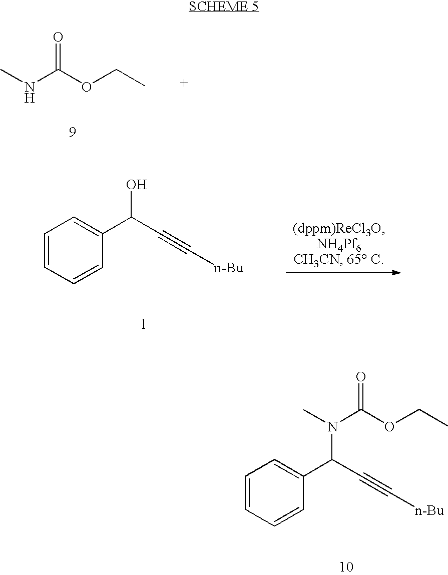

The reaction of Scheme 5 was carried out as follows:

1-Phenyl-2-heptyn-1-ol (1) (100 mg, 0.53 mmol) was added to a 1 dram vial with a threaded cap containing three equivalents of N-methyl ethyl carbamate (9) (165 mg, 1.59 mmol). To the vial was then added (dppm)ReCl₃O (18.5 mg, 0.027 mmol, 5 mol %) and ammonium hexafluorophosphate (4.5 mg, 0.027 mmol, 5 mol %) in a solution of 0.5 ml acetonitrile. The resulting green mixture was stirred at 65° C. for three hours. Upon completion, the reaction mixture was chromatographed directly on a silica gel column (5:1 hexanes:ether) to afford 10 as a clear oil (93%). ¹H NMR (CDCl₃, 300 MHz) δ 7.53 (m, 2H), 7.37-7.25 (m, 3H), 6.41-6.23 (br. d, 1H, indicative of rotational conformers about carbamate C-N bond), 4.20 (m, 2H), 2.71 (s, 3H), 2.31 (td, 2H, J=6.9 Hz, 2.4 Hz), 1.61-1.39 (m, 4H), 1.30 (t, 3H, J=7.2 Hz), 0.96 (t, 3H, 9.0 Hz).

### EXAMPLE 22

### Propargyl Arylation

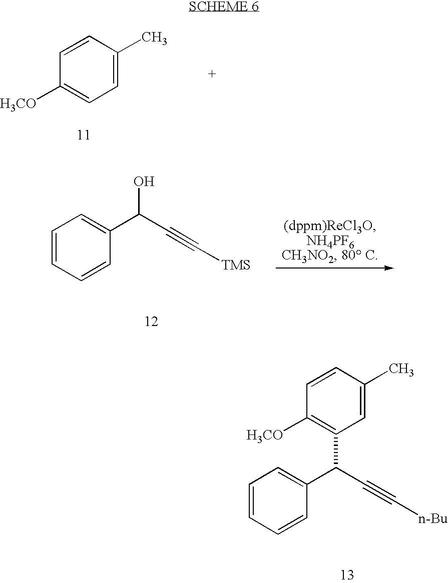

The reaction of Scheme 6 was carried out as follows:

In a medium sized scintillation vial equipped with a stir bar was added propargyl alcohol 12 (400 mg, 1.96 mmol), 4-methylanisole (11) (493 μL, 3.91 mmol), nitromethane (4 mL), (dppm)ReCl₃O (65 mg, 5 mole %) and NH₄PF₆ (15 mg, 2.5 mol %). This mixture was then heated to 80° C. for 5 h. Upon completion, the mixture was cooled to ambient temperature and all volatiles were removed. The residue was redissolved in a small amount of CH₂Cl₂ and chromatographed on silica gel (1:99 EtOAc / Hexanes) to give a mixture of 13 and 4-methylanisole (˜570 mg). This material was then left in-vacuo for ˜24 h to give 13 (532 mg, 88%).

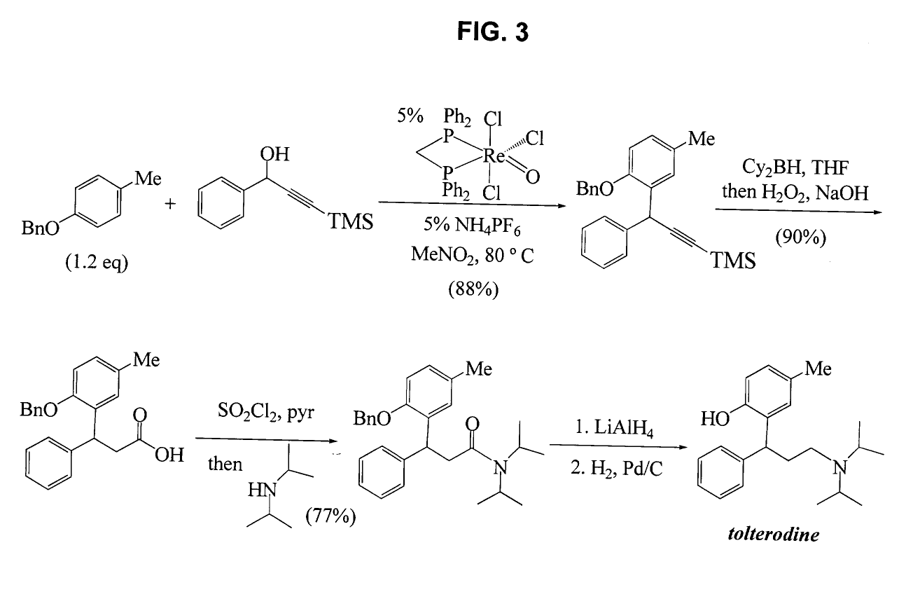

FIG. 3 illustrates a modification of this reaction that was carried out to provide the pharmaceutical agent tolterodine, a drug known for the treatment of urinary incontinence. The synthesis illustrates how the substituted alkynes prepared using the method of the invention are used in the organic synthesis of a commercially significant compound.

### EXAMPLE 23

### Propargyl Arylation with a Ketal-substituted Nucleophile

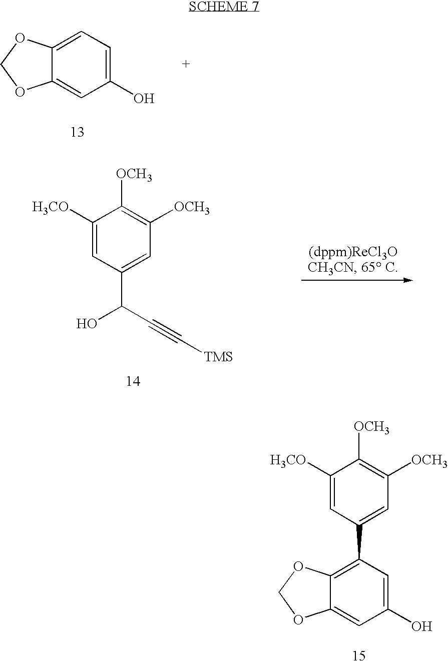

The reaction of Scheme 7 was carried out as follows:

In a medium sized scintillation vial equipped with a stir bar was added propargyl alcohol 13 (500 mg, 1.70 mmol), sesamol 14 (258 mg, 1.87 mmol), acetonitrile (3.4 mL), and (dppm)ReCl₃O (˜1 mg, ˜0.1 mol %). This mixture was then heated to 65° C. for 4 h. Upon completion, the mixture was cooled to ambient temperature where 15 (405 mg, 57%) precipitated and was collected by filtration. All volatiles were then removed from the mother liquor, then redissolved in a small amount of CH₂Cl₂ and chromatographed on silica gel (1:10 to 1:6 EtOAc/Hexanes) to give additional 15 (160 mg, 23%, for a total of 565 mg, 80%).

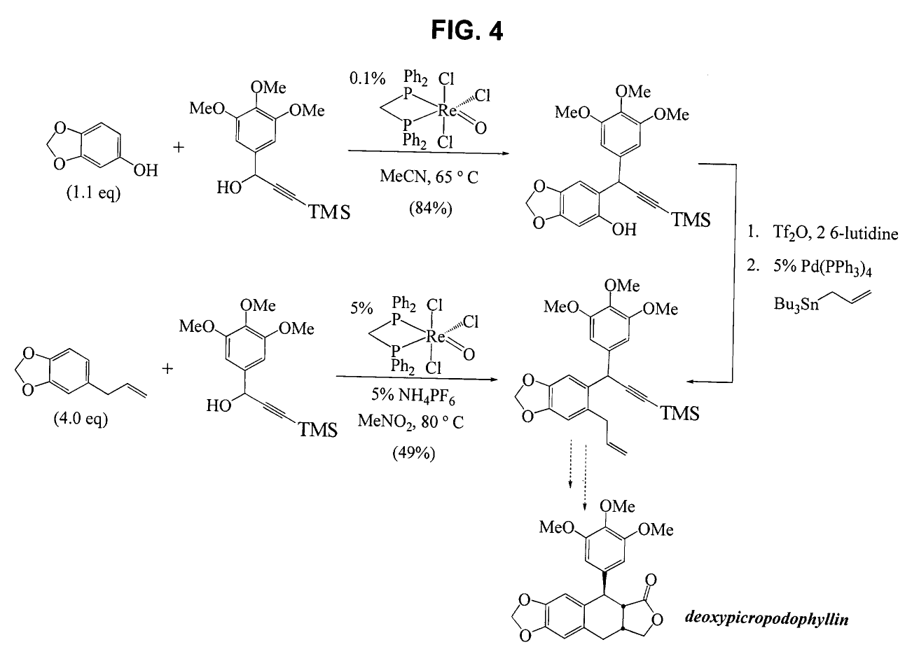

FIG. 4 illustrates a modification of this reaction that was carried out to provide the pharmaceutical agent deoxypicropodophyllin, an antineoplastic drug. The synthesis further illustrates how the substituted alkynes prepared using the method of the invention are used in the organic synthesis of a commercially significant compound.

### EXAMPLE 24

### Propargylation with an Allylsilane as Nucleophile

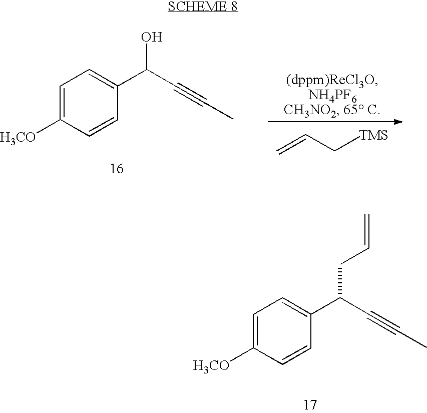

The reaction of Scheme 8 was carried out as follows:

1-(4-methoxyphenyl)-2-butyn-1-ol (16) (100 mg, 0.57 mmol) was added to a 1 dram vial with a threaded cap containing three equivalents of allyltrimethylsilane (195 mg, 1.7 mmol). To the vial was then added (dppm)ReCl₃O (16.7 mg, 0.023 mmol, 4 mol %) in a solution of 2.3 ml nitromethane (0.25 M of the propargylic alcohol). The resulting green mixture was stirred at 65° C., with the course of the reaction being monitored at intervals by thin layer chromatography. After completion, the reaction mixture was concentrated in vacuo and chromatographed directly. Flash chromatography eluting with hexanes afforded the allyl adduct 17 (109 mg, 96%) as a pale yellow oil.

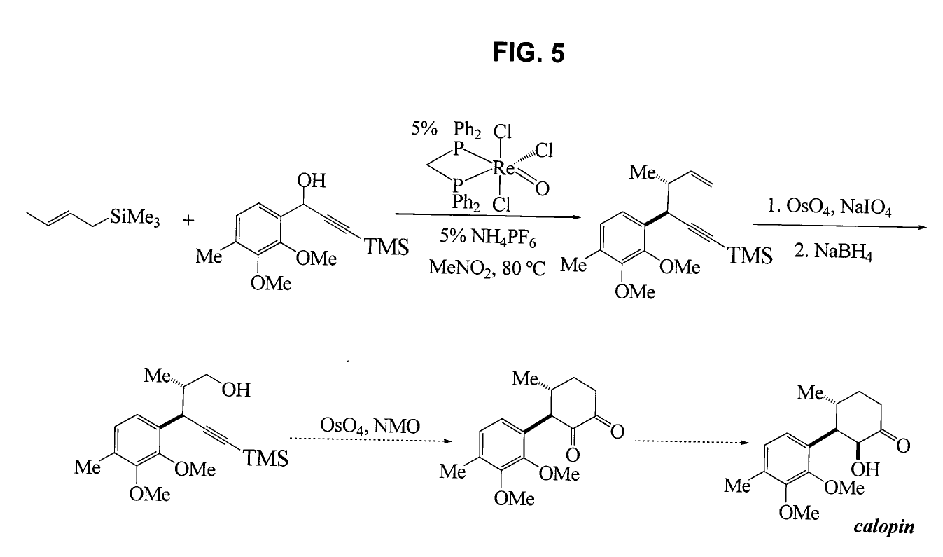

FIG. 5 illustrates a modification of this reaction that was carried out to provide the pharmaceutical agent calopin. The synthesis provides an additional example of how the substituted alkynes prepared using the method of the invention are used in the organic synthesis of a commercially significant compound.

### EXAMPLE 25

### Catalyst Recovery

The reaction of Example 24 was repeated, except that the reaction was run at room temperature. After completion and concentration, the resulting green oil was dissolved in methylene chloride. The catalyst was precipitated and recovered upon addition of hexanes (82% recovered).

### EXAMPLE 26

### Synthesis of Rhenium (V) Phenoxy-oxazolidine Complexes

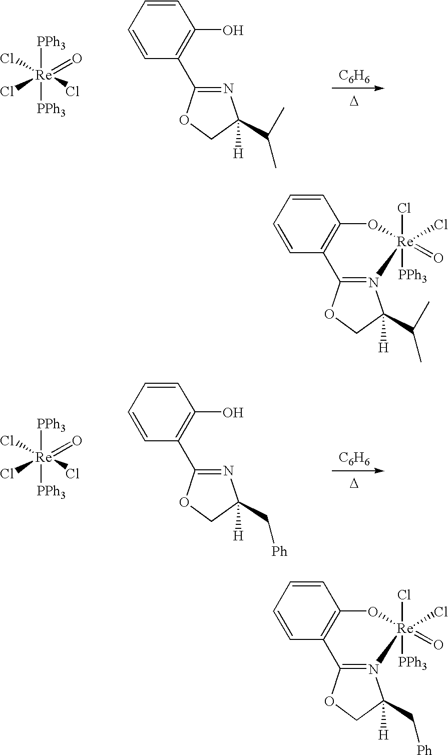

To a clear solution of the 2-(2-hydroxyphenyl)-(4S)-isopropyloxazolidine (3.7 g, 18.0 mmol) in benzene (150 mL), at reflux, was added bis(triphenylphosphine)oxorhenium(V) trichloride (1.5 g, 1.80 mmol). The resulting green solution was refluxed for 2 h, cooled to room temperature and concentrated to approximately 50 mL. The green precipitate was collected and washed with diethyl ether (3×50 mL), to afford the chiral rhenium complex (1.10 g, 83%) as a green solid. ¹H-NMR (CD₂Cl₂): δ 7.60-7.37 (m, 19H), 7.12 (ddq, J=8.2, 7.1 and 1.8 Hz, 1H), 6.91 (td, J=7.1 and 1.8 Hz, 1H), 6.63 (dd, J=8.2 and 0.8 Hz), 4.48 (dd, J=9.8 and 4.3 Hz, 1H), 3.96 (t, J=9.8 Hz, 1H), 3.57 (ddd, J=9.8, 4.0 and 2.8 Hz, lH), 2.92 (m, 1H), 1.00 (d, J=6.6 Hz, 3H), 0.82 (d, J=7.1 Hz, 3H). ³¹P-NMR (CD₂Cl₂): −18.5. An analogous procedure was carried out to synthesize the benzyloxazolidine analogue using 2-(2-hydroxyphenyl)-(4S)-benzyloxazolidine as a starting material.

### EXAMPLE 27

### Synthesis of Chiral Bisphosphine Rhenium (V) Complexes

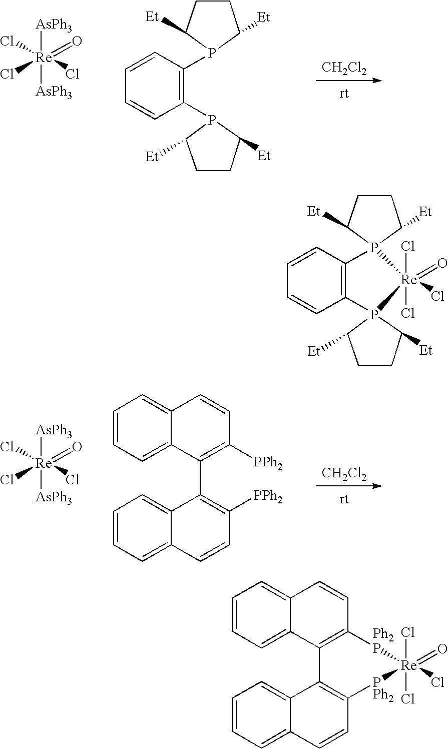

To a yellow suspension of bis(triphenylarsine)oxorhenium(V) trichloride (1.3 g, 1.41 mmol) in methylene chloride (40 mL) was added 1,2-bis((2S,5S)-2,5-diethylphospholano) benzene ((S,S)-Et-DUPHOS, obtained from Strem Chemicals Inc., Newburyport, Mass.) (500 mg, 1.38 mmol), as shown in the first scheme above. The resulting green reaction mixture was stirred at room temperature for 10 h, then filtered to remove some white precipitate. The filtrate was concentrated to approximately 10 mL and then diluted with diethyl ether (150 mL). The precipitated green solid was collected and washed with diethyl ether (3×50 mL) to afford the desired complex (1.08 g, 81%) as a green solid. ¹H-NMR (CD₂Cl₂): δ 8.0 (m, 2H), 7.8 (m, 2H), 3.0-2.0 (m, 8H), 2.0-1.0 (m, 24H). ³¹P-NMR (CD₂Cl₂): 40.20, 31.73. An analogous procedure was used to prepare the rhenium complex shown in the second scheme above, substituting 2,2′-bis(diphenylphosphino)-1,1′-binaphthyl (BINAP) for ((S,S)-Et-DUPHOS.

1. A method for catalytically modifying an alkyne reactant substituted at the propargylic position with a leaving group capable of displacement by a nucleophilic reactant, comprising contacting such an alkyne reactant with a nucleophilic reactant in the presence of a catalytically effective amount of a rhenium (V) oxo complex having at least one electron donor ligand and at least two anionic ligands, under reaction conditions effective to provide for nucleophilic displacement of the leaving group.

2. The method of claim 1, wherein the alkyne reactant has the structure of formula (I) 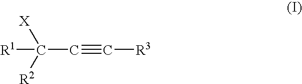
in which:
X is the leaving group;
R¹ is selected from hydrogen, C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl;
R² is selected from hydrogen, C₁-C₂₄ hydrocarbyl, substituted C₁-C₂₄ hydrocarbyl, heteroatom-containing C₁-C₂₄ hydrocarbyl, substituted heteroatom-containing C₁-C₂₄ hydrocarbyl, and functional groups; and
R³ is selected from hydrogen, silyl, C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl.

3. The method of claim 2, wherein:
R¹ is hydrogen or lower hydrocarbyl;
R² and R³ are independently selected from hydrogen, C₁-C₂₄ alkyl, C₁-C₂₄ heteroalkyl, C₅-C₂₄ aryl, C₅-C₂₄ heteroaryl, C₆-C₂₄ alkaryl, C₆-C₂₄ heteroalkaryl, C₆-C₂₄ aralkyl, and C₆-C₂₄ heteroaralkyl, any of which, with the exception of hydrogen, may be substituted.

4. The method of claim 3, wherein:
R² and R³ are independently selected from hydrogen, C₁-C₁₂ alkyl, C₁-C₁₂ heteroalkyl, C₅-C₁₄ aryl, C₅-C₁₄ heteroaryl, C₆-C₁₆ alkaryl, C₆-C₁₆ heteroalkaryl, C₆-C₁₆ aralkyl, and C₆-C₁₆ heteroaralkyl, any of which, with the exception of hydrogen, may be substituted.

5. The method of claim 2, wherein X is selected from —OH, —OR⁴, —SH, and —SR⁵, wherein R⁴ and R⁵ are selected from C₁-C₂₄ hydrocarbyl, substituted C₁-C₂₄ hydrocarbyl, heteroatom-containing C₁-C₂₄ hydrocarbyl, substituted heteroatom-containing C₁-C₂₄ hydrocarbyl, and activating groups that promote the displacement of X by the nucleophilic reactant.

6. The method of claim 5, wherein X is selected from —OH and —OR⁴, wherein R⁴ is C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, or substituted C₅-C₁₄ heteroaryl.

7. The method of claim 6, wherein X is —OH.

8. The method of claim 1, wherein the nucleophilic reactant is a compound comprising a nucleophilic group selected from hydroxyl, hydrocarbyloxy, primary amino, secondary amino, silyl, alkenyl, aryl, and heteroaryl, any of which, with the exception of hydroxyl, may be further substituted and/or heteroatom-containing.

9. The method of claim 8, wherein the nucleophilic reactant is selected from:
R⁶—OH, wherein R⁶ is selected from selected from C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl;
R⁷—O—R⁸, wherein R⁷ and R⁸ are defined as for R⁶, and further wherein R⁷ and R⁸ may be linked to form a cyclic ether;
R⁹—NH—R¹⁰, wherein R⁹ is selected from selected from C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl, and R¹⁰ is selected from hydrogen, C₁-C₂₄ hydrocarbyl, substituted C₁-C₂₄ hydrocarbyl, heteroatom-containing C₁-C₂₄ hydrocarbyl, substituted heteroatom-containing C₁-C₂₄ hydrocarbyl, amine-protecting groups, and functional groups, and further wherein R⁹ and R¹⁰ may be linked to form a cyclic amine;
R¹¹—Si(R¹²R¹³R¹⁴), wherein R¹¹ is hydrogen, cyano, cyanato, azido, or boronato, and R¹², R¹³ and R¹⁴ are independently selected from selected from C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl;
R¹⁵R¹⁶C═CR¹⁷R¹⁸ wherein R¹⁵ is an electron-donating substituent, and R¹⁶, R¹⁷, and R¹⁸ are selected from hydrogen, C₁-C₂₄ hydrocarbyl, substituted C₁-C₂₄ hydrocarbyl, heteroatom-containing C₁-C₂₄ hydrocarbyl, and substituted heteroatom-containing C₁-C₂₄ hydrocarbyl, and further wherein any two of R¹⁶, R¹⁷, and R¹⁸ may be linked to form a cyclic olefin; and
Ar(R¹⁹)^{m} wherein Ar is C₅-C₂₄ aryl, substituted C₅-C₂₄ aryl, C₅-C₂₄ heteroaryl, or substituted C₅-C₂₄ heteroaryl, R¹⁹ is an electron-donating substituent, and m is at least 1, wherein, when m is 2 or more, the R¹⁹ substituents may be the same or different.

10. The method of claim 7, wherein the nucleophilic reactant is a compound comprising a nucleophilic group selected from hydroxyl, hydrocarbyloxy, primary amino, secondary amino, silyl, alkenyl, aryl, and heteroaryl, any of which, with the exception of hydroxyl, may be further substituted and/or heteroatom-containing.

11. The method of claim 10, wherein the nucleophilic reactant is selected from:
R⁶—OH, wherein R⁶ is selected from C₁-C₁₂ alkyl, substituted C₁-C₁₂ alkyl, C₁-C₁₂ heteroalkyl, substituted C₁-C₁₂ heteroalkyl, C₁-C₁₂ alkenyl, substituted C₁-C₁₂ alkenyl, C₁-C₁₂ heteroalkenyl, substituted C₁-C₁₂ heteroalkenyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl;
R⁷—O—R⁸, wherein R⁷ and R⁸ are defined as for R⁶, and further wherein R⁷ and R⁸ may be linked to form a cyclic ether;
R⁹—NH—R¹⁰, wherein R⁹ is selected from C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl, and R¹⁰ is selected from hydrogen, C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-Cl₆ heteroalkaryl, substituted C₆-C₁₆ heteroalkaryl, C₆-C₁₆ aralkyl, substituted C₆-C₁₆ aralkyl, C₆-C₁₆ heteroaralkyl, substituted C₆-C₁₆ heteroaralkyl, C₂-C₁₂ alkoxycarbonyl, substituted C₂-C₁₂ alkoxycarbonyl, C₆-C₁₄ aryloxycarbonyl, and further wherein R⁹ and R¹⁰ may be linked to form a five-or six-membered N-heterocycle optionally substituted and/or containing additional heteroatoms;
H—Si(R¹²R¹³R¹⁴), wherein R¹², R¹³ and R¹⁴ are independently selected from selected from C₁-C₁₂ alkyl and C₅-C₁₄ aryl;
R¹⁵R¹⁶C═CR¹⁷R¹⁸ wherein R¹⁵ is an electron-donating substituent, and R¹⁶, R¹⁷, and R¹⁸ are selected from hydrogen, C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl, and further wherein any two of R¹⁶, R¹⁷, and R¹⁸ may be linked to form a five- or six-membered cyclic olefin; and
Ar(R¹⁹)^{m}, wherein Ar is C₅-C₁₄ aryl, substituted C₅-C₁₄ aryl, C₅-C₁₄ heteroaryl, or substituted C₅-C₁₄ heteroaryl, R¹⁹ is an electron-donating substituent, and m is 1 or 2, wherein, when m is 2, the R¹⁹ substituents may be the same or different.

12. The method of claim 11, wherein the nucleophilic reactant is R⁶—OH.

13. The method of claim 1, wherein the rhenium (V) oxo complex has the structure of formula (II) 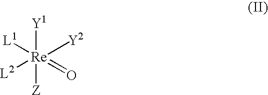
wherein:
L¹ and L² are monodentate neutral electron donor ligands, or may be taken together to form a single bidentate neutral electron donor ligand;
Y¹ and Y² are anionic ligands; and
Z is a monodentate neutral electron donor ligand or an anionic ligand.

14. The method of claim 13, wherein:
L¹ and L² are independently selected from the group consisting of phosphine, sulfonated phosphine, phosphite, phosphinite, phosphonite, arsine, stibine, ether, amine, amide, imine, sulfoxide, carboxyl, nitrosyl, pyridine, substituted pyridine, imidazole, substituted imidazole, pyrazine, and thioether, or L¹ and L² together form a bidentate ligand in which at least one coordinating heteroatom is other than N; and
Y¹ and Y² are selected from hydride, halide, C₁-C₂₄ alkyl, C₅-C₂₄ aryl, C₁-C₂₄ alkoxy, C₅-C₂₄ aryloxy, C₃-C₂₄ alkyldiketonate, C₅-C₂₄ aryldiketonate, C₂-C₂₄ alkoxycarbonyl, C₅-C₂₄ aryloxycarbonyl, C₂-C₂₄ acyl, C₁-C₂₄ alkylsulfonato, C₅-C₂₄ arylsulfonato, C₁-C₂₄ alkylsulfanyl, C₅-C₂₄ arylsulfanyl, C₁-C₂₄ alkylsulfinyl, or C₅-C₂₄ arylsulfinyl, any of which, with the exception of hydride and halide, are optionally further substituted with one or more groups selected from halide, C₁-C₆ alkyl, C₁-C₆ alkoxy, and phenyl.

15. The method of claim 14, wherein Z is a monodentate neutral electron donor ligand

16. The method of claim 15, wherein Z is selected from the group consisting of phosphine, sulfonated phosphine, phosphite, phosphinite, phosphonite, arsine, stibine, ether, amine, amide, imine, sulfoxide, carboxyl, nitrosyl, pyridine, substituted pyridine, imidazole, substituted imidazole, pyrazine, and thioether.

17. The method of claim 14, wherein Z is an anionic ligand.

18. The method of claim 17, wherein Z is selected from hydride, halide, C₁-C₂₄ alkyl, C₅-C₂₄ aryl, C₁-C₂₄ alkoxy, C₅-C₂₄ aryloxy, C₃-C₂₄ alkyldiketonate, C₅-C₂₄ aryldiketonate, C₂-C₂₄ alkoxycarbonyl, C₅-C₂₄ aryloxycarbonyl, C₂-C₂₄ acyl, C₁-C₂₄ alkylsulfonato, C₅-C₂₄ arylsulfonato, C₁-C₂₄ alkylsulfanyl, C₅-C₂₄ arylsulfanyl, C₁-C₂₄ alkylsulfinyl, or C₅-C₂₄ arylsulfinyl, any of which, with the exception of hydride and halide, are optionally further substituted with one or more groups selected from halide, C₁-C₆ alkyl, C₁-C₆ alkoxy, and phenyl.

19. The method of claim 14, wherein:
L¹ and L² are independently selected from phosphines of the formula P(R²⁰)₃, where each R²⁰ is independently monocyclic aryl, C₁-C₁₀ alkyl, substituted C₁-C₁₀ alkyl, substituted monocyclic aryl, or C₁-C₁₀ alkyl; and
Y¹ and Y² are selected from halide and lower alkoxy.

20. The method of claim 19, wherein L¹ and L² are selected from tricyclohexylphosphine, tricyclopentylphosphine, triphenylphosphine, tri(m-tolyl)phosphine, tri(p-tolyl)phosphine, 1 and cyclohexyldiphenylphosphine; and
Y¹ and Y² are halide.

21. The method of claim 20, wherein Z is selected from tricyclohexylphosphine, tricyclopentylphosphine, triphenylphosphine, tri(m-tolyl)phosphine, tri (p-tolyl)phosphine, and cyclohexyldiphenylphosphine.

22. The method of claim 20, wherein Z is halide or lower alkoxy.

23. The method of claim 14, wherein:
L¹ and L² together form a bidentate ligand in which at least one coordinating heteroatom is other than N; and
Y¹ and Y² are selected from halide and lower alkoxy; and
Z is selected from halide, lower alkoxy, tricyclohexylphosphine, tricyclopentylphosphine, triphenylphosphine, tri(m-tolyl)phosphine, tri(p-tolyl)phosphine, and cyclohexyldiphenylphosphine.

24. The method of claim 23, wherein the complex has the structure of formula (III) 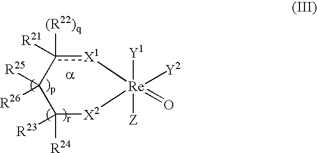
wherein:
α is an optional double bond;
p is zero, 1, or 2;
q is zero when α is present, and q is 1 when α is absent;
r is zero or 1;
X¹ is selected from O, P(R²⁷R²⁸), and NR²⁹ wherein R²⁷, R²⁸, and R²⁹ are independently selected from C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl, and when X¹ is N, then α is present;
X² is selected from O and P(R^{27A}R^{28A}), wherein R^{27A} and R^{28A} are defined as for R²⁷ and R²⁸, respectively;
R²¹, R²², R²³, and R²⁴ are independently selected from hydrogen, C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl;
R²⁵ and R²⁶ are independently selected from hydrogen, C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C_{14 5}-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, substituted C₆-C₁₆ heteroaralkyl, and functional groups, and further wherein any two or more of R²¹, R²², R²³, R²⁴; R²⁵, R²⁶ R²⁷, R²⁸, and R²⁹ may be linked to form a cyclic group.

25. The method of claim 24, wherein α is absent, X¹ and X² are O, p is 1, q is zero, r is 1, and R²⁴ and R²⁶ are hydrogen, such that the complex has the structure of formula (IV)

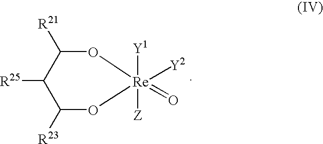

26. The method of claim 25, wherein R²¹, R²³, and R²⁵ are hydrogen, Y¹ and Y² are halide, and Z is halide, tricyclohexylphosphine, tricyclopentylphosphine, or triphenylphosphine.

27. The method of claim 24, wherein α is present, X¹ is NR²⁹, X² is O, p is 1, q is zero, r is 1, R²⁴ and R²⁶ are hydrogen, such that the complex has the structure of formula (V)

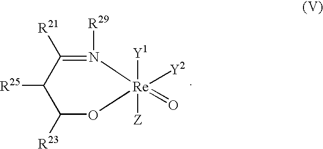

28. The method of claim 27, wherein R²³ and R²⁵ are linked to form a phenyl group and R²¹ and R²⁹ are linked to form a 4,5-dioxazole ring, such that the complex has the structure of formula (VI) 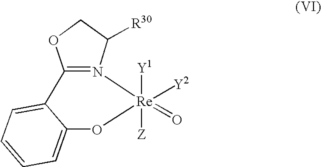
wherein R³⁰ is selected from hydrogen, C₁-C₁₂ alkyl, phenyl, and benzyl.

29. The method of claim 28, wherein Y¹ and Y² are halide, and Z is halide, tricyclohexylphosphine, tricyclopentylphosphine, or triphenylphosphine.

30. The method of claim 24, wherein α is absent, q is 1, X¹ is PR²⁷R²⁸, X² is PR^{27A}R^{28A}, and R²⁷, R²⁸, R^{27A}, and R^{28A} are aryl.

31. The method of claim 30, wherein r and p are zero.

32. The method of claim 31, wherein R²¹ and R²² are hydrogen.

33. The method of claim 30, wherein r is 1 and p are zero.

34. The method of claim 33, wherein R²¹, R²², R²³, and R²⁴ are hydrogen.

35. The method of claim 30, wherein r and p are 1.

36. The method of claim 35, wherein R²¹, R²², R²³, R²⁴, R²⁵, and R²⁶ are hydrogen.

37. The method of claim 32, wherein R²⁷, R²⁸, R^{27A}, and R^{28A} are phenyl, such that the complex is (dppm)ReO(Y¹Y²Z).

38. The method of claim 34, wherein R²⁷, R²⁸, R^{27A}, and R^{28A} are phenyl, such that the complex is (dppe)ReO(Y¹Y²Z).

39. The method of claim 36, wherein R²⁷, R²⁸, R^{27A}, and R^{28A} are phenyl, such that the complex is (dppp)ReO(Y¹Y²Z).

40. The method of claim 23, wherein the complex has the structure of formula (VII) 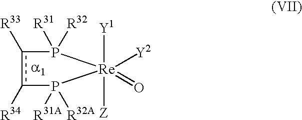
wherein:
α₁ is an optional double bond; and
R³¹, R³², R^{31A}, R^{32A}, R³³, and R³⁴ are independently selected from C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl, and wherein any two or more of R³¹, R³², R^{31A}, R^{32A}, R³³, and R³⁴ may be taken together to form a cyclic group.

41. The method of claim 40, wherein α₁ is absent.

42. The method of claim 41, wherein R³³ and R³⁴ are hydrogen.

43. The method of claim 42, wherein R³¹, R³², R^{31A}, and R^{32A} are aryl.

44. The method of claim 43, wherein R³¹, R³², R^{31A}, and R^{32A} are phenyl.

45. The method of claim 40, wherein α₁ is present.

46. The method of claim 45, wherein R³³ and R³⁴ taken together are phenyl or naphthalenyl.

47. The method of claim 46, wherein R³¹, R³², R^{31A}, and R^{32A} are aryl.

48. The method of claim 46, wherein R³¹, R³², R^{31A}, and R^{32A} are phenyl.

49. The method of claim 23, wherein the complex has the structure of formula (VIII) 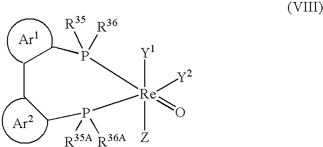
in which:
Ar¹ and Ar² are independently selected from C₅-C₂₄ aryl, substituted C₅-C₂₄ aryl, C₅-C₂₄ heteroaryl, and substituted C₅-C₂₄ heteroaryl; and
R³⁵, R³⁶, R^{35A}, and R^{36A} are independently selected from C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl, and wherein any two or more of R³¹, R³², R^{31A}, R^{32A}, R³³, and R³⁴ may be taken together to form a cyclic group.

50. The method of claim 49, wherein R³⁵, R³⁶, R³⁵A, and R^{36A} are aryl.

51. The method of claim 50, wherein R³⁵, R³⁶, R³⁵A, and R^{36A} are phenyl.

52. The method of claim 49, wherein Ar¹ and Ar² are phenyl or naphthalenyl.

53. The method of claim 51, wherein Ar¹ and Ar² are phenyl or naphthalenyl.

54. The method of claim 23, wherein the complex has the structure of formula (IX) 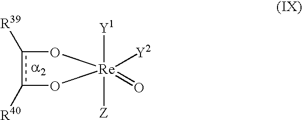
wherein:
α₂ is an optional double bond; and
R³⁹ and R⁴⁰ are independently selected from C₁-C₁₂ alkyl, substituted C₁-C₁₂ alkyl, C₁-C₁₂ heteroalkyl, substituted C₁-C₁₂ heteroalkyl, C₅-C₁₄ aryl, substituted C₅-C₁₄ aryl, C₅-C₁₄ heteroaryl, substituted C₅-C₁₄ heteroaryl, C₆-C₁₆ alkaryl, substituted C₆-C₁₆ alkaryl, C₆-C₁₆ heteroalkaryl, substituted C₆-C₁₆ heteroalkaryl, C₆-C₁₆ aralkyl, substituted C₆-C₁₆ aralkyl, C₆-C₁₆ heteroaralkyl, and substituted C₆-C₁₆ heteroaralkyl, and wherein R³⁹ and R⁴⁰ may be taken together to form a cyclic group.

55. The method of claim 54, wherein α₂ is present and R³⁹ and R⁴⁰ taken together form a phenyl ring.

56. The method of claim 1, wherein the contacting is carried out in a reaction mixture that additionally includes a co-catalyst.

57. The method of claim 56, wherein the co-catalyst is a salt of a cation that can abstract an anionic ligand and an anion that does not coordinate to the rhenium atom within the complex.

58. The method of claim 1, wherein the comprised of a alkyne reactant is additionally contacted with the complex is positively charged and associated with a negatively charged counterion.

59. The method of claim 1, wherein the molar ratio of the nucleophilic reactant to the alkyne reactant is greater than 1:1.

60. The method of claim 59, wherein the molar ratio of the nucleophilic reactant to the alkyne reactant is in the range of about 1.5:1 to about 3:1.

61. The method of claim 1, wherein the reaction conditions comprise carrying out said contacting in a polar aprotic solvent at a temperature in the range of about 20° C. to about 80° C.

62. The method of claim 61, wherein said contacting is carried out at a temperature in the range of about 20° C. to about 25° C.

63. The method of claim 62, wherein following completion of the reaction, the catalyst is recovered by: adding a nonpolar solvent to the reaction mixture in an amount sufficient to result in precipitation of the catalyst; and removing the precipitated catalyst from the reaction mixture.

64. A method for synthesizing a propargyl ether, comprising contacting an alkyne reactant substituted at the propargylic position with a hydroxyl group with an alcohol in the presence of a catalytically effect amount of a complex having the structure of formula (X) 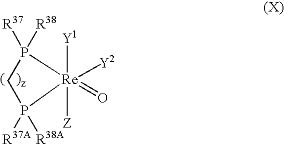
wherein R³⁷, R³⁸, R^{37A}, and R^{38A} are aryl, z is 1, 2, or 3, and Y¹, Y², and Z are anionic ligands.

65. The method of claim 64, wherein R³⁷, R³⁸, R^{37A}, and R^{38A} are phenyl and Y¹, Y², and Z are halide.

66. The method of claim 65, wherein the alcohol is a monohydric alcohol.

67. A method for synthesizing an enone comprising contacting a propargylic alcohol with a catalytically effective amount of a rhenium (V) oxo complex having at least one electron donor ligand and at least two anionic ligands, under reaction conditions effective to effect rearrangement of the alcohol to an enone.
